(19) United States
(12) Patent Application Publication
(10) Pub. No.: US 2021/0252015 A1
(43) Pub. Date: Aug. 19, 2021

(54) CERTAIN (2S)-N-[(1S)-1-CYANO-2-PHENYLETHYL]-1,4-OXAZEPANE-2-CARBOXAMIDES FOR TREATING INFLAMMATORY BOWEL DISEASE

(71) Applicant: Insmed Incorporated, Bridgewater, NJ (US)

(72) Inventor: Jimin ZHANG, Bridgewater, NJ (US)

(21) Appl. No.: 17/260,602

(22) PCT Filed: Jul. 16, 2019
(86) PCT No.: PCT/US2019/042026

§ 371 Date: Jan. 15, 2021

Related U.S. Application Data
(60) Provisional application No. 62/699,491, filed on Jul. 17, 2018.

(51) Int. Cl. A61K 31/553; A61K 9/00; A61K 45/06; A61P 1/04; A61K 9/08; A61K 47/38; C07K 16/24

(52) U.S. Cl. CPC A61K 31/553; A61K 9/0053; A61K 9/0019; A61K 45/06; C07K 16/241; A61K 9/08; A61K 47/38; C07K 16/244; A61P 1/04

(57) ABSTRACT

The present disclosure relates to methods for treating inflammatory bowel disease, with compositions comprising an effective amount of certain (2S)—N-[(1S)-1-cyano-2-phenylethyl]-1,4-oxazepane-2-carboxamide compounds of Formula (I), including pharmaceutically acceptable salts thereof, that inhibit dipeptidyl peptidase 1 (DPP1) activity. In one embodiment, the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide.

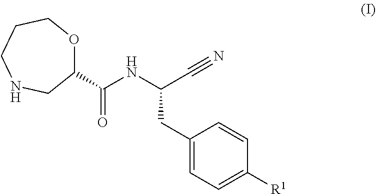

### CROSS REFERENCE TO RELATED APPLICATION

This application claims priority from U.S. Provisional Application Ser. No. 62/699,491, filed Jul. 17, 2018, the disclosure of which is incorporated by reference herein in its entirety for all purposes.

### BACKGROUND OF THE INVENTION

Inflammatory bowel disease (IBD) is a group of inflammatory conditions that affect the colon and small intestine. The most common IBDs are Crohn's disease and ulcerative colitis.¹ The symptoms of Crohn's disease and ulcerative colitis are similar with patients usually presenting with abdominal pain and diarrhea. Crohn's disease can affect both ileum and colon while ulcerative colitis usually affects only the innermost lining of the colon and rectum.²

The development of IBD animal models has contributed to the understanding and development of treatments for IBD.³ The dextran sulfate sodium (DSS)-induced model of colitis in C57BL/6 mice (DSS-Induced Model) is used to study acute colitis. The adoptive transfer of sorted naïve T-cells into RAG2^{-/-} mice (Adoptive T-Cell Model⁴) is one of the best-characterized immunological animal models of chronic colitis and allows for the focused and detailed examination of T cell-mediated pathological mechanisms. The DSS-Induced and Adoptive T-Cell Models generally respond well to established clinical therapies, including the anti-p40 monoclonal antibodies ustekinumab (marketed in the United States as Stelara®).

There is currently no cure for IBD. Current IBD therapies include anti-inflammatory drugs and immune system modulators, which manage patient symptoms by, for example, relieving symptoms, inducing symptom remission and preventing symptom relapse. Anti-inflammatory drugs (such as sulfasalazine, mesalamine and corticosteroids) are first line medications to induce remission of IBD symptoms⁵ and immune system suppressors are generally used to maintain IBD symptom remission.⁶ The most common immune system modulators used to treat IBD are azathioprine, mercaptopurine and biological therapies such as anti-tumor necrosis factor monoclonal antibodies (anti-TNFα, such as infliximab, adalimumab and certolizumab pegol) and anti-p40 monoclonal antibodies (such as ustekinumab).

The present invention addresses the need for a therapy effective for the treatment of inflammatory bowel disease.

### SUMMARY OF THE INVENTION

In one aspect, a method is provided for treating inflammatory bowel disease (IBD) in a patient need thereof. The method comprises, in one embodiment, administering to the patient in need of IBD treatment, a pharmaceutical composition comprising an effective amount of a compound of formula (I), or a pharmaceutically acceptable salt thereof.

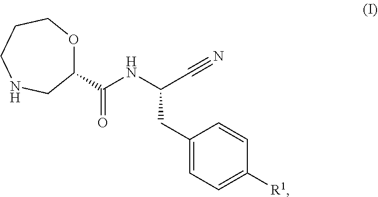

wherein,

### R¹ is

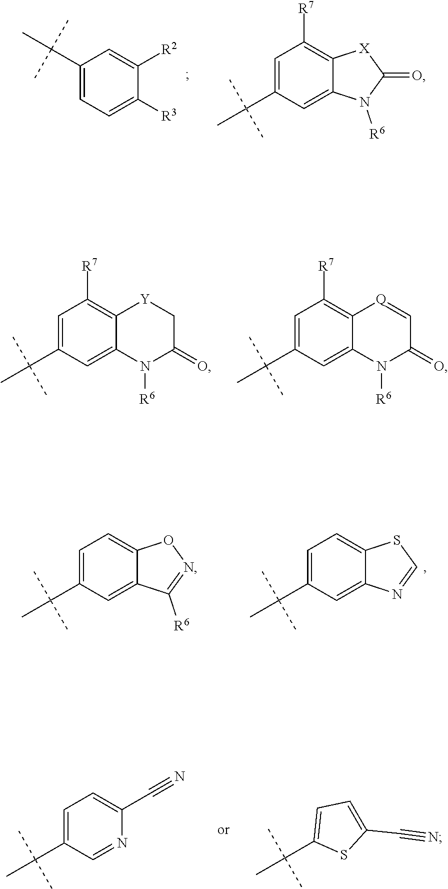

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R₅ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;

### X is O, S or CF₂;

### Y is O or S; and

### Q is CH or N.

In one embodiment of the method for treating IBD in a patient in need thereof, the pharmaceutical composition comprises an effective amount of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, (also referred to herein as INS1007),

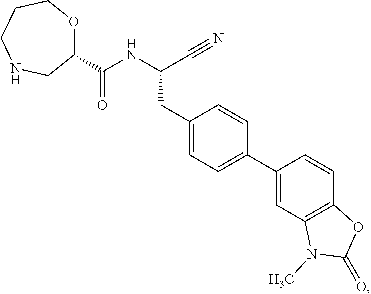 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

Administration routes include oral administration. Administration schedules can be determined by the user of the method, e.g., a prescribing physician. In one embodiment, administration is once daily. In another embodiment, administration is twice daily. In another embodiment, administration 1× daily, once every other day, once every third day, once every fourth day, 2× weekly, 3× weekly or 4× weekly.

In one embodiment of the method for treating IBD, the IBD is ulcerative colitis.

In one embodiment of the method for treating IBD, the IBD is Crohn's disease.

### BRIEF DESCRIPTION OF THE FIGURES

FIG. 1 shows the study design for the DSS-induced colitis murine study described in Example 1A.

FIG. 2A is a graph of the mean endoscopy score at Day 10 of the DSS-induced colitis murine study described in Example 1A. FIG. 2B is a graph of the mean stool score at Day 10 of the DSS-induced colitis murine study described in Example 1A. **P≤0.01; ***P≤0.001; ****P≤0.0001 vs. vehicle DSS control (Group 4) as measured by Dunnett's multiple comparisons test with one-way ANOVA.

FIG. 3A is a graph of the mean endoscopy score at Day 14 of the DSS-induced colitis murine study described in Example 1A. FIG. 3B is a graph of the mean stool score at Day 14 of the DSS-induced colitis murine study described in Example 1A. **P≤0.01; ***P≤0.001; vs. vehicle DSS control (Group 4) as measured by Dunnett's multiple comparisons test with one-way ANOVA.

FIG. 4A is a graph of the mean endoscopy score at Day 21 of the DSS-induced colitis murine study described in Example 1A. FIG. 4B is a graph of the mean stool score at Day 214 of the DSS-induced colitis murine study described in Example 1A. *P≤0.05; **P≤0.01 vs. vehicle DSS control (Group 4) as measured by Dunnett's multiple comparisons test with one-way ANOVA.

FIG. 5 is a graph of the calculated mean body weight loss area under the curve (AUC) for Days 0 to 21 of the DSS-induced colitis murine study described in Example 1A. ****P≤0.0001 vs. vehicle DSS control (Group 4) as measured by Dunnett's multiple comparisons test with one-way ANOVA.

FIG. 6 shows the study design for the DSS-induced colitis murine study described in Example 1B.

FIG. 7A is a graph of the mean endoscopy score at Day 10 of the DSS-induced colitis murine study described in Example 1B. FIG. 7B is a graph of the mean stool score at Day 10 of the DSS-induced colitis murine study described in Example 1B. *P≤0.05; ****P≤0.001 vs. vehicle DSS control (Group 2) as measured by Dunnett's multiple comparisons test with one-way ANOVA.

FIG. 8A is a graph of the mean endoscopy score at Day 14 of the DSS-induced colitis murine study described in Example 1B. FIG. 8B is a graph of the mean stool score at Day 14 of the DSS-induced colitis murine study described in Example 1B. *P≤0.05; ***P<0.005 vs. vehicle DSS control (Group 2) as measured by Dunnett's multiple comparisons test with one-way ANOVA.

FIG. 9 is a graph of the calculated mean body weight loss area under the curve (AUC) for Days −7 to 21 of the DSS-induced colitis murine study described in Example 1B. *P<0.05; **P<0.01 vs. vehicle DSS control (Group 2) as measured by Dunnett's multiple comparisons test with one-way ANOVA.

FIG. 10 is a graph of neutrophil activity (ng/μg protein) derived from the bone marrow of mice in the DSS-induced colitis murine study described in Example 1A. *P<0.05 vs. vehicle DSS control (Group 2) as measured by Dunnett's multiple comparisons test with one-way ANOVA.

FIG. 11A is a graph of the average mean endoscopy score at Day 10 of the DSS-induced colitis murine study described in Examples 1A and 1B. FIG. 11B is a graph of the average mean stool score at Day 10 of the DSS-induced colitis murine study described in Examples 1A and 1B. *P<0.05 vs. vehicle DSS control (Group 2) as measured by Dunnett's multiple comparisons test with one-way ANOVA.

FIG. 12A is a graph of the average mean endoscopy score at Day 10 of the DSS-induced colitis murine study described in Examples 1A and 1B. FIG. 12B is a graph of the average mean stool score at Day 10 of the DSS-induced colitis murine study described in Examples 1A and 1B. *P<0.05 vs. vehicle DSS control (Group 2) as measured by Dunnett's multiple comparisons test with one-way ANOVA.

FIG. 13 is a graph of the average mean body weight change on day 21 of the DSS-induced colitis murine study described in Examples 1A and 1B. *P<0.05 vs. vehicle DSS control (Group 2) as measured by Dunnett's multiple comparisons test with one-way ANOVA.

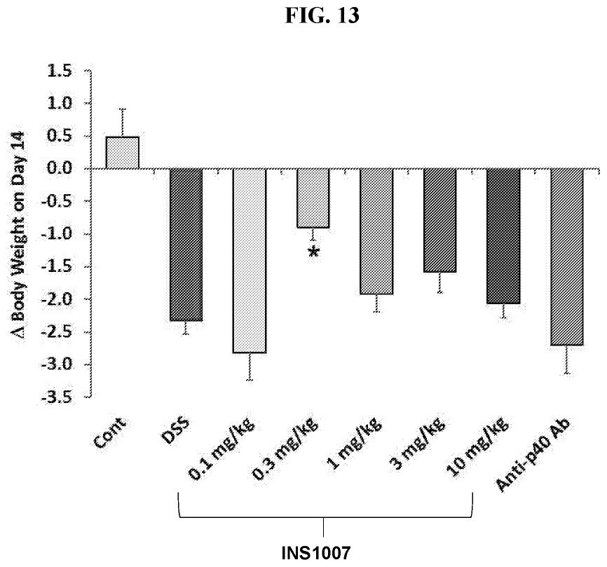

FIG. 14 shows the study design for the murine Adoptive T cell transfer model study described in Example 2.

FIG. 15 is a graph of the percent survival (Days 0 to 42) for the murine Adoptive T cell transfer model study described in Example 2. *P<0.05 vs. vehicle control (Group 2) as measured by log-rank (Mantel-Cox) test.

FIG. 16A is a graph of the mean endoscopy score at Day 28 of the murine Adoptive T cell transfer model study described in Example 2. FIG. 16B is a graph of the mean stool score at Day 28 of the murine Adoptive T cell transfer model study described in Example 2. *P<0.05 vs. vehicle T-cell induced control (Group 2) as measured by Dunnett's multiple comparisons test with one-way ANOVA.

FIG. 17A is a graph of the mean endoscopy score at Day 42 of the murine Adoptive T cell transfer model study described in Example 2. FIG. 17B is a graph of the mean stool score at Day 42 of the murine Adoptive T cell transfer model study described in Example 2. *P<0.05 vs. vehicle T-cell induced control (Group 2) as measured by Dunnett's multiple comparisons test with one-way ANOVA.

FIG. 18 is a graph of the calculated mean body weight loss area under the curve (AUC) for Days 0 to 42 for surviving mice of the murine Adoptive T cell transfer model study described in Example 2.

FIG. 19 is a graph of the calculated mean body weight loss area under the curve (AUC) for Days 0 to 42 of the murine Adoptive T cell transfer model study described in Example 2.

### DETAILED DESCRIPTION OF THE INVENTION

As used herein, “C₁₋₃” means a carbon group having 1, 2 or 3 carbon atoms.

The term “alkyl”, unless otherwise noted, includes both straight and branched chain alkyl groups and may be substituted or non-substituted. “Alkyl” groups include, but are not limited to, methyl, ethyl, n-propyl, i-propyl, butyl, pentyl.

The the term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Provided herein are methods for treating IBD patients via administration of a pharmaceutical composition comprising an effective amount of a compound of formula (I), or a pharmaceutically acceptable salt thereof.

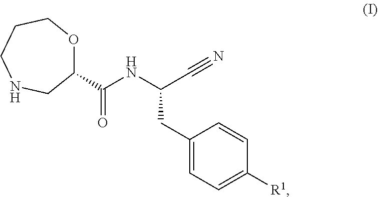

wherein,

### R¹ is

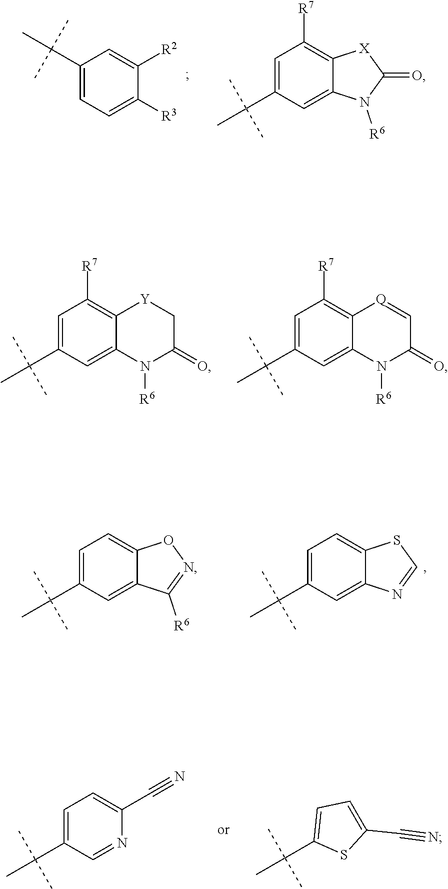

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;

### X is O, S or CF₂;

### Y is O or S; and

### Q is CH or N.

In one embodiment R¹ is

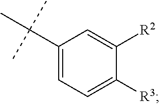

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring.

In a further embodiment, R¹ is

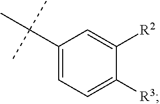

R² is hydrogen, F, Cl or C₁₋₃alkyl; and R³ is hydrogen, F, Cl, CN or SO₂C₁₋₃alkyl.

In still a further embodiment, R¹ is

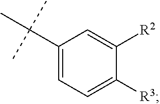

R² is hydrogen, F or C₁₋₃alkyl; and R³ is hydrogen, F or CN.

In another embodiment, R¹ is

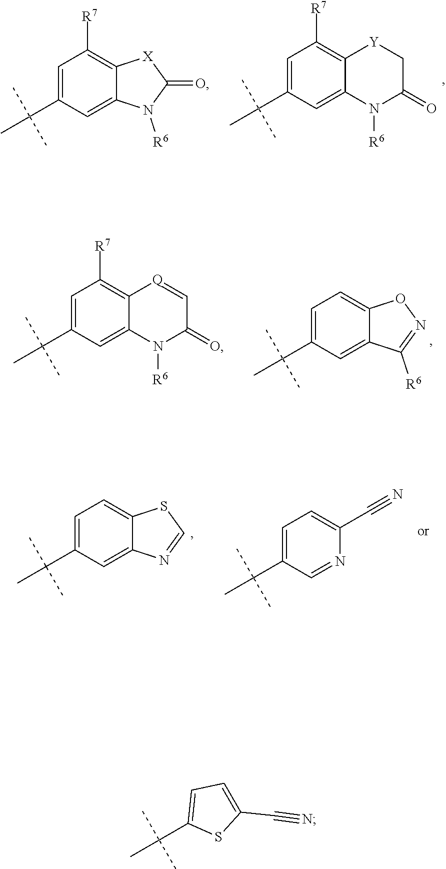

X is O, S or CF₂; Y is O or S; Q is CH or N; R⁶ is C₁₋₃alkyl, wherein the C₁₋₃alkyl is optionally substituted by 1, 2 or 3 F and/or optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

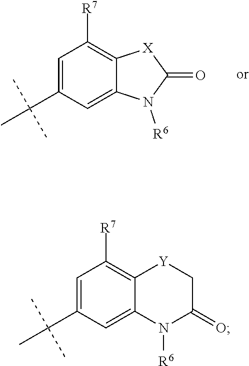

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

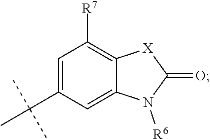

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In still a further embodiment, R¹ is

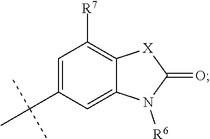

X is O; R⁶ is C₁₋₃alkyl, wherein the C₁₋₃alkyl is optionally substituted by 1, 2 or 3 F; and R⁷ is hydrogen.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is selected from hydrogen, F, Cl, CN or SO₂C₁₋₃alkyl.

In still a further embodiment, R³ is selected from hydrogen, F or CN.

In one embodiment, R⁶ is C₁₋₃alkyl, wherein said C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran.

In a further embodiment, R⁶ is C₁₋₃alkyl, wherein said C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment of the methods provided herein, the composition administered to the patient comprises an effective amount of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide (INS1007):

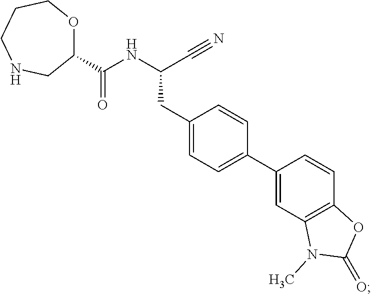 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the compound of formula (I) is:
(2S)—N-[(1S)-1-Cyano-2-(4′-cyanobiphenyl-4-yl)ethyl]-1,4-oxazepane-2-carboxamide,
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)—N-{(1S)-1-Cyano-2-[4-(3-methyl-1,2-benzoxazol-5-yl)phenyl]ethyl}-1,4-oxazepane-2-carboxamide,
(2S)—N-{(1S)-1-Cyano-2-[4′-(trifluoromethyl)biphenyl-4-yl]ethyl}-1,4-oxazepane-2-carboxamide,
(2S)—N-[(1S)-1-Cyano-2-(3′,4′-difluorobiphenyl-4-yl)ethyl]-1,4-oxazepane-2-carboxamide,
(2S)—N-{(1S)-1-Cyano-2-[4-(6-cyanopyridin-3-yl)phenyl]ethyl}-1,4-oxazepane-2-carboxamide,
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide,
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)—N-{(1S)-1-Cyano-2-[4-(5-cyanothiophen-2-yl)phenyl]ethyl}-1,4-oxazepane-2-carboxamide,
(2S)—N-[(1S)-2-(4′-Carbamoyl-3′-fluorobiphenyl-4-yl)-1-cyanoethyl]-1,4-oxazepane-2-carboxamide,
(2S)—N-{(1S)-1-Cyano-2-[4-(1-methyl-2-oxo-1,2-dihydroquinolin-7-yl)phenyl]ethyl}-1,4-oxazepane-2-carboxamide,
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)—N-{(1S)-1-Cyano-2-[4′-(methylsulfonyl)biphenyl-4-yl]ethyl}-1,4-oxazepane-2-carboxamide,
(2S)—N-{(1S)-2-[4′-(Azetidin-1-ylsulfonyl)biphenyl-4-yl]-1-cyanoethyl}-1,4-oxazepane-2-carboxamide,
(2S)—N-[(1S)-1-Cyano-2-(4′-fluorobiphenyl-4-yl)ethyl]-1,4-oxazepane-2-carboxamide,
(2S)—N-{(1S)-2-[4-(1,3-Benzothiazol-5-yl)phenyl]-1-cyanoethyl}-1,4-oxazepane-2-carboxamide, or
(2S)—N-[(1S)-1-Cyano-2-(4′-cyanobiphenyl-4-yl)ethyl]-1,4-oxazepane-2-carboxamide,

or a pharmaceutically acceptable salt of one of the foregoing compounds.

Formula I, its subgenuses, and specific compounds of Formula (I), including INS1007, as well as methods of making the same, are disclosed in U.S. Pat. No. 9,522,894, the disclosure of which is incorporated by reference in its entirety for all purposes.

The IBD treatment methods provided herein comprise the administration of a composition comprising an effective amount of a compound of formula (I), or a pharmaceutically acceptable salt thereof, to a patient in need of IBD treatment. The compounds of formula (I) and their pharmaceutically acceptable salts are inhibitors of dipeptidyl peptidase 1 (DPP1) activity. In one embodiment, the compound is INS1007, or a pharmaceutically acceptable salt thereof.

Administration routes include oral administration. Administration schedules can be determined by the user of the method, e.g., a prescribing physician. In one embodiment, administration is once daily. In another embodiment, administration is twice daily. In another embodiment, administration 1× daily, once every other day, once every third day, once every fourth day, 2× weekly, 3× weekly or 4× weekly.

The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression of the state, disorder or condition or at least one of its clinical or subclinical symptoms).

In one embodiment of the method of treatments provided herein, the IBD is Crohn's disease.

In one embodiment of the method of treatments provided herein, treating a patient in need thereof comprises decreasing Crohn's Disease Activity Index (CDAI) for the patient, as compared to the CDAI score prior to treatment (W. Best, et al., Development of a Crohn's Disease Activity Index, National Cooperative Crohn's Disease Study, Gastroenterology, 1976, vol. 70, pages 439-444, incorporated by reference herein in its entirety for all purposes). In a further embodiment, a composition comprising an effective amount of a compound of Formula (I) is administered orally. In a further embodiment, the treating comprises decreasing the patient's CDAI score by at least about 50 points, at least about 100 points, at least about 150 points, at least about 200 points or at least about 250 points as compared to the patient's CDAI score prior to treatment. In a further embodiment, the treating comprises decreasing the patient's CDAI score to less than about 200 points, less than about 150 points, less than about 100 points or less than about 50 points. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

The CDAI uses a 7-day patient diary to establish a numeric value that correlates to disease severity. A CDAI score of at least 150 indicates a patient in active Crohn's disease. A CDAI score of at least 450 indicates a patient in with very severe Crohn's disease.

The CDAI score is calculated by the sum of:
(1) 7-day sum of number of liquid or very soft stools times two (i.e., scaling factor);
(2) 7-day sum of abdominal pain (0=none, 1=mild, 2=moderate, 3=severe) times five;
(3) 7-day sum of general well-being (0=generally well, 1=slightly under par, 2=poor, 3=very poor, 4=terrible) times seven;
(4) number of symptoms manifested at time of survey (the 6 surveyed symptoms are arthritis/arthralgia, iritis/uveitis; erythema nodosum/pyoderma gangrenosum/aphthous stomatitis, anal fissure, fistula or abscess, other fistula, fever over 100° F. during last week) times twenty;
(5) taking lomitil/opiates for diarrhea (0=no, 1=yes) times thirty;
(6) abdominal mass (0=none; 2=questionable; 5=definite) times ten;
(7) hematocrit (males: 47-patient hematocrit; females: 42-patient hematocrit) times six; and
(8) percent below standard weight (nomogram).

In one embodiment of the method of treatments provided herein, treating a patient in need thereof comprises treating a patient with moderate to severe Crohn's disease. In yet a further embodiment, treating in need thereof comprises treating a patient with a CDAI score greater than 220 and less or equal to 440.

In one embodiment of the method of treatments provided herein, treating a patient with Crohn's comprises decreasing Harvey Bradshaw Index score for the patient, as compared to the Harvey Bradshaw Index score prior to treatment (R. Harvey, et al., A Simple Index of Crohn's Disease Activity, Lancet, 1980, vol. 1, 514, incorporated by reference herein in its entirety for all purposes). In a further embodiment, the patient's initial Harvey Bradshaw Index store is reduced by 1, 2, 3, 4, 5, 6, 7, 8, 9, 10, 11, 12, 13, 14, or 15. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

The Harvey Bradshaw Index survey uses a patient worksheet to establish a numeric value that correlates to disease severity. A Harvey Bradshaw Index score of less than 5 indicates Remission; between 5-7 indicates Mild Disease; between 8-16 indicates Moderate Disease and greater than 16 indicates Severe Disease.

The Harvey Bradshaw Index score is calculated by the sum of:
(1) Patient's general well-being (for the previous day; 0=very well, 1=slightly below par, 2=poor, 3=very poor, 4=terrible);
(2) abdominal pain (0=none, 1=dubious, 2=definite, 3=definite and tender);
(3) number of stools per day (for the previous day);
(4) abdominal mass (0=none; 1=dubious; 2=definite; 3=definite and tender)
(5) number of symptoms manifested at time of survey (the 8 surveyed symptoms are arthralgia, uveitis; erythema nodosum, aphthous ulcers, pyoderma gangrenosum, anal fissure, new fistula abscess).

In one embodiment of the method of treatments provided herein, treating in need thereof comprises treating a patient with moderate to severe Crohn's disease. In yet a further embodiment, treating in need thereof comprises treating a patient with an initial Harvey Bradshaw Index score greater than 7.

In one embodiment of the method of treatments provided herein, treating a patient in need thereof comprises decreasing Simplified Endoscopy Score for Crohn's Disease (SES-CD), as compared to the SES-CD score prior to treatment (M. Daperno, et al., Development and validation of a new, simplified endoscopic activity score for Crohn's disease: the SES-CD, Gastrointest Endosc. 2004 October; 60(4):505-12, incorporated by reference herein in its entirety for all purposes). In a further embodiment, the treating comprises decreasing the patient's SES-CD to less than 5, less than 4, less than 3, less than 2, less than 1, or to 0. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

A patient's SES-CD for Crohn's Disease is determined by a physician and evaluates the endoscopic parameters ulcer size, ulcerated and affected surfaces and stenosis scored 0 to 3 as described in M. Daperno 2004. The SES-CD score range is 0-56. A SES-CD score of 0-2 indicates Remission; between 3-6 indicates mild endoscopic activity; between 7-15 indicates moderate endoscopic activity and greater than or equal to 16 indicates severe endoscopic activity.

In one embodiment of the method of treatments provided herein, the IBD is ulcerative colitis.

In one embodiment of the method of treatments provided herein, treating a patient in need thereof comprises decreasing total Mayo Score for the patient, as compared to the total Mayo Score prior to treatment (K. Schroeder, et al., Coated oral 5-aminosalicylic acid therapy for mildly to moderately active ulcerative colitis. A randomized study, N Engl J Med, 1987; 317:1625-9, incorporated by reference herein in its entirety for all purposes). In a further embodiment, the treating comprises decreasing the patient's total Mayo Score by at least 2 points, at least 3 points, or at least 4 points, as compared to the patient's total Mayo Score prior to treatment. In one embodiment, decreasing is by about 10%, about 20%, about 25%, about 30%, about 40%, about 50%, about 60%, about 70%, or about 80% of the patient's total Mayo Score as compared to the patient's total Mayo Score prior to treatment. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

The Mayo Score survey uses the sum four subscores to establish a numeric value that correlates to disease severity. The Mayo Subscores include stool frequency subscore, rectal bleeding subscore, physician's global assessment and endoscopy subscore. In a further embodiment, the treating comprises decreasing the at least one of the patient's Mayo Subscores by at least 1 points or at least 2 points, as compared to the patient's Mayo Subscore prior to treatment.

In one embodiment of the method of treatments provided herein, treating in need thereof comprises treating a patient with moderate to severe ulcerative colitis (UC). In yet a further embodiment, treating in need thereof comprises treating a patient with a total Mayo score greater than 6. In yet a further embodiment, treating in need thereof comprises treating a patient with a Mayo endoscopy subscore of greater than 2.

In one embodiment of the method of treatments provided herein, treating a patient in need thereof comprises decreasing total Ulcerative Colitis Disease Activity Index (UCDAI) for the patient, as compared to the total UCDAI score prior to treatment (L. Sutherland, et al., 5-Aminosalicylic Acid Enema in the Treatment 551 of Distal Ulcerative Colitis, Proctosigmoiditis, and Proctitis, Gastroenterology, 1987, 92:1894-8., incorporated by reference herein in its entirety for all purposes). In a further embodiment, the treating comprises decreasing the patient's total UCDAI score to less than about 2 or less than 1. In one embodiment, decreasing is by about 10%, about 20%, about 25%, about 30%, about 40%, about 50%, about 60%, about 70%, or about 80% of the patient's total UCDAI Score as compared to the patient's total UCDAI Score prior to treatment. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

The UCDAI survey uses the sum four subscores to establish a numeric value that correlates to disease severity. The UCDAI Subscores include stool frequency subscore, rectal bleeding subscore, physician's rating of disease activity and mucosal appearance subscore. In a further embodiment, the treating comprises decreasing the at least one of the patient's UCDAI Subscores by at least 1 points or at least 2 points, as compared to the patient's UCDAI Subscore prior to treatment.

In one embodiment of the method of treatments provided herein, treating UC in a patient in need thereof comprises mucosal healing of the patient's Ulcerative Colitis as compared to prior to treatment. In a further embodiment, the treating comprises mucosal healing indicated by a decrease the patient's Mayo endoscopy subscore to one or less. In a further embodiment, the treating comprises mucosal healing indicated by a decrease the patient's histological grade to zero according to the Geboes histological assessment described in K. Geboes, et al., A Reproducible Grading Scale for Histological Assessment of Inflammation in Ulcerative Colitis, Gut, 200, 47, 404-409, hereby incorporated by reference in its entirety for all purposes.

In one embodiment of the method of treatments provided herein, treating IBD in a patient in need thereof comprises achieving remission of the patient's IBD. In a further embodiment, treating IBD patient in need thereof comprises achieving sustained remission of the patient's IBD. In a further embodiment, treating IBD patient provides remission of the patient's IBD for at least 1 week, at least two weeks, at least one month, at least two months, at least three months, at least 4 months, at least 5 months or at least 6 months. In a further embodiment, treating an IBD patient provides remission of the patient's IBD as indicated by the treating providing a patient FCP concentration of less than 250 μg/g. In a further embodiment, treating an IBD patient provides remission of the patient's IBD as indicated by the treating providing a patient CRP concentration of less than 5 mg/kL. In a further embodiment, treating a Crohn's patient provides remission of the patient's Crohn's disease as indicated by the treating providing a patient CDAI score of less than 150 points. In a further embodiment, treating a Crohn's patient provides remission of the patient's Crohn's disease as indicated by the treating providing a patient Harvey Bradshaw Index score of less than 4 points. In a further embodiment, treating a UC patient provides remission of the patient's UC as indicated by the treating providing a patient total Mayo score of less than or equal to 2 points with no subscore greater than 1 point.

In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily.

In another embodiment of the method for treating IBD provided herein, a composition comprising an effective amount of a compound of formula (I), or a pharmaceutically acceptable salt thereof, is administered to a patient in need thereof. The method comprises decreasing intra-leukocyte proteinase 3 (PR3) activity, as compared to the patient's intra-leukocyte PR3 activity, prior to treatment. The compound of formula (I) in a further embodiment, is INS1007, or a pharmaceutically acceptable salt thereof. In one embodiment, the compound of formula (I) is administered orally to the patient in need of treatment. In yet a further embodiment, administration is 1× daily, once every other day, once every third day, once every fourth day, 2× weekly, 3× weekly or 4× weekly. In even a further embodiment, administration of the compound is once daily.

In one embodiment, the PR3 activity is measured in leukocytes obtained from the patient's whole blood. In one embodiment, decreasing is by about 10%, about 20%, about 25%, about 30%, about 40%, about 50%, about 60%, about 70%, about 80%. In another embodiment, decreasing PR3 blood activity comprises decreasing by at least about 1%, at least about 5%, at least about 10%, at least about 20%, at least about 30%, at least about 40%, at least about 50%, at least about 60%, at least about 70% or at least about 80%. In one embodiment, the compound of formula (I) is INS1007, or a pharmaceutically acceptable salt thereof. In yet a further embodiment, administration is 1× daily, once every other day, once every third day, once every fourth day, 2× weekly, 3× weekly or 4× weekly. In even a further embodiment, administration of the compound is once daily via oral administration.

In another embodiment of the method of treatments provided herein, treating a patient in need thereof comprises administering to the patient a compound of Formula (I), or a pharmaceutically acceptable salt thereof, and decreasing the neutrophil cell surface expression of proteinase 3 of the patient, as compared to the neutrophil cell surface expression of proteinase 3 prior to treatment. In one embodiment, decreasing comprises decreasing the PR3 cell surface expression by about 10%, about 20%, about 25%, about 30%, about 40%, about 50%, about 60%, about 70%, about 80%. In another embodiment, decreasing proteinase 3 cell surface expression comprises decreasing by at least about 1%, at least about 5%, at least about 10%, at least about 20%, at least about 30%, at least about 40%, at least about 50%, at least about 60%, at least about 70% or at least about 80%. In one embodiment, the compound of formula (I) is INS1007, or a pharmaceutically acceptable salt thereof. In a further embodiment, the compound of formula (I) is administered orally. In even a further embodiment, the compound of formula (I) is administered once daily.

In another embodiment of the method for treating IBD provided herein, a composition comprising an effective amount of a compound of formula (I), or a pharmaceutically acceptable salt thereof, is administered to a patient in need of IBD treatment, wherein the method comprises decreasing the neutrophil serine protease (NSP) activity in the patient's blood, as compared to the patient's NSP activity, prior to treatment. In one embodiment, the compound of formula (I) is administered via oral administration. In one embodiment, administration is once daily. In another embodiment, administration is twice daily. In another embodiment, administration 1× daily, once every other day, once every third day, once every fourth day, 2× weekly, 3× weekly or 4× weekly. The compound of formula (I) in one embodiment, is INS1007, or a pharmaceutically acceptable salt thereof. The NSP is neutrophil elastase (NE), proteinase 3 (PR3) and/or cathepsin G (CatG). In one embodiment, decreasing NSP activity is by about 10%, about 20%, about 25%, about 30%, about 40%, about 50%, about 60%, about 70%, about 80%. In another embodiment, decreasing NSP activity comprises decreasing NSP activity by at least about 1%, at least about 5%, at least about 10%, at least about 20%, at least about 30%, at least about 40%, at least about 50%, at least about 60%, at least about 70% or at least about 80%.

In yet another embodiment of the method for treating IBD provided herein, a composition comprising an effective amount of a compound of formula (I), or a pharmaceutically acceptable salt thereof, is administered to a patient in need thereof, wherein the method comprises decreasing the patient's c-reactive protein (CRP) blood concentration, as compared to the patient's CRP blood concentration prior to treatment. In one embodiment, the compound of formula (I) is administered via oral administration. The compound of formula (I) in one embodiment is INS1007, or a pharmaceutically acceptable salt thereof. In one embodiment, the CRP blood concentration is measured in the patient's blood plasma or blood serum. In a further embodiment, administration is 1× daily, once every other day, once every third day, once every fourth day, 2× weekly, 3× weekly or 4× weekly.

In one embodiment, the method comprises decreasing the CRP blood concentration of the patient by about 10%, about 20%, about 25%, about 30%, about 40%, about 50%, about 60%, about 70%, about 80%. In another embodiment, decreasing CRP blood concentration comprises decreasing by at least about 1%, at least about 5%, at least about 10%, at least about 20%, at least about 30%, at least about 40%, at least about 50%, at least about 60%, at least about 70% or at least about 80%. In one embodiment, the CRP blood concentration is measured in the patient's blood plasma or blood serum.

In yet another embodiment of the method for treating IBD provided herein, a composition comprising an effective amount of a compound of formula (I), or a pharmaceutically acceptable salt thereof, is administered to a patient in need thereof, wherein the method comprises decreasing the patient's fecal calprotectin (FCP), as compared to the patient's FCP concentration prior to treatment. In one embodiment, the compound of formula (I) is administered via oral administration. The compound of formula (I) in one embodiment is INS1007, or a pharmaceutically acceptable salt thereof. In one embodiment, the FCP concentration is measured in the patient's feces. In a further embodiment, administration is 1× daily, once every other day, once every third day, once every fourth day, 2× weekly, 3× weekly or 4× weekly.

In one embodiment, the method comprises decreasing the FCP concentration of the patient by about 10%, about 20%, about 25%, about 30%, about 40%, about 50%, about 60%, about 70%, about 80%. In another embodiment, decreasing FCP concentration comprises decreasing by at least about 1%, at least about 5%, at least about 10%, at least about 20%, at least about 30%, at least about 40%, at least about 50%, at least about 60%, at least about 70% or at least about 80%. In one embodiment, the FCP concentration is measured in the patient's feces.

In one embodiment of the method of treatments provided herein, treating a patient in need thereof comprises treating a patient with moderate to severe IBD. In yet a further embodiment, treating a patient in need thereof comprises treating a patient with a FCP concentration greater than about 150 μg/g, about 200 μg/g, about 250 μg/g, or about 300 μg/g greater. In yet a further embodiment, treating a patient in need thereof comprises treating a patient with a CRP concentration greater than about 3 mg/dL, about 4 mg/dL, about 5 mg/dL, about 6 mg/dL or about 7 mg/dL.

The dosage administered will vary with the compound employed, the mode of administration, the treatment desired and the disorder indicated. In one embodiment, if the compound is administered orally, then the daily dosage of the compound of the disclosure may be in the range from 0.01 micrograms per kilogram body weight (μg/kg) to 100 milligrams per kilogram body weight (mg/kg).

In one embodiment, the compound of Formula (I) is administered in an oral dosage form. In a further embodiment, the compound of Formula (I) is administered as a 10 mg to 50 mg dosage form, for example, a 5 mg dosage form, a 10 mg dosage form, a 15 mg dosage form, a 20 mg dosage form, a 25 mg dosage form, a 30 mg dosage form, 35 mg dosage form, a 40 mg dosage form, a 45 mg dosage form or a 50 mg dosage form. In a further embodiment, the dosage form is a 25 mg or 40 mg dosage form. In a further embodiment, the dosage form is administered once daily. In even a further embodiment, the compound is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide (INS1007), or a pharmaceutically acceptable salt thereof.

The compounds of formula (I), or pharmaceutically acceptable salts thereof, may be used on their own but will generally be administered in the form of a pharmaceutical composition in which the formula (I) compound/salt (active ingredient) is in a composition comprising a pharmaceutically acceptable adjuvant(s), diluents(s) and/or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes.

Depending on the mode of administration, the pharmaceutical composition will comprise from 0.05 to 99% w (percent by weight), for example, from 0.05 to 80% w, or from 0.10 to 70% w, or from 0.10 to 50% w, of active ingredient, all percentages by weight being based on total composition.

In one oral administration embodiment, the oral dosage form is a film-coated oral tablet. In a further embodiment, the dosage form is an immediate release dosage form with rapid dissolution characteristics under in vitro test conditions.

In one embodiment, the oral dosage form is administered once daily. In a further embodiment, the oral dosage form is administered at approximately the same time every day, e.g., prior to breakfast. In another embodiment, the composition comprising an effective amount of formula (I) is administered 2× day. In yet another embodiment, the composition comprising an effective amount of formula (I) is administered once-a-week, every other day, every third day, 2× week, 3× week, 4× week, or 5× week.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also liquid or semisolid formulations of the compound of the disclosure may be filled into hard gelatine capsules.

In one embodiment, the composition is an oral disintegrating tablet (ODT). ODTs differ from traditional tablets in that they are designed to be dissolved on the tongue rather than swallowed whole

In one embodiment, the composition is an oral thin film or an oral disintegrating film (ODF). Such formulations, when placed on the tongue, hydrate via interaction with saliva, and releases the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral application may be in the form of syrups, solutions or suspensions. Solutions, for example, may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

A compound of formula (I), or a pharmaceutically acceptable salt thereof, may also be administered in conjunction with one or more additional active agents used for the treatment of IBD via one of the methods described herein.

The one or more additional active agents is administered concurrently, sequentially or in admixture with a compound of Formula (I), for the treatment of IBD.

In one embodiment, the one or more additional active agents is an anti-inflammatory drug. In even a further embodiment, the anti-inflammatory drug is an aminosalicyclate.

The one or more additional active agents, in one embodiment, is sufasazine.

The one or more additional active agents, in one embodiment, is mesalamine.

The one or more additional active agents, in one embodiment, is balsalazide.

The one or more additional active agents, in one embodiment, is olsalazine.

In yet another embodiment, the one or more additional active agents, is a steroid. In a further embodiment, the steroid is a corticosteroid. In even a further embodiment, the further compound is a glucocorticoid.

The one or more additional active agents, in one embodiment, is budesonide.

The one or more additional active agents, in one embodiment, is prednisone.

In one embodiment, the one or more additional active agents is an immune system modulator. In even a further embodiment, the immune system modulator is one or more thiopurine, an anti-tumor necrosis factor α (TNF-α) monoclonal antibody, and an anti-p40 monoclonal antibody.

The one or more additional active agents, in one embodiment, is azathioprine

The one or more additional active agents, in one embodiment, is 6-mercaptopurine

The one or more additional active agents, in one embodiment, is methotrexate.

The one or more additional active agents, in one embodiment, is ustekinumab.

The one or more additional active agents, in one embodiment, is infliximab.

The one or more additional active agents, in one embodiment, is adalimumab.

The one or more additional active agents, in one embodiment, is certolizumab pegol.

The one or more additional active agents, in one embodiment, is infliximab.

In one combination therapy embodiment, the compound of the disclosure, or a pharmaceutically acceptable salt thereof, is administered concurrently or sequentially with one or more further active ingredients selected from one or more of those provided above. For example, the compound of Formula (I), or a pharmaceutically acceptable salt thereof, may be administered concurrently or sequentially with a further pharmaceutical composition for use as a medicament for the treatment of IBD. The further pharmaceutical composition may be a medicament which the patient may already be prescribed (e.g., an existing standard or care medication), and may itself be a composition comprising one or more active ingredients selected from those defined above.

A compound of formula (I) or a pharmaceutically acceptable salt thereof can be synthesized by reacting a compound of formula (II),

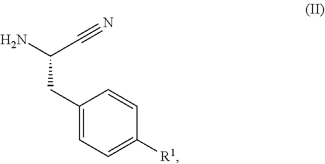

wherein R¹ is as defined in formula (I), with a compound of formula (III),

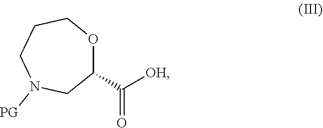

wherein PG represents a protecting group (e.g. tert-butoxycarbonyl), and optionally thereafter carrying out one or more of the following procedures:
converting a compound of formula (I) into another compound of formula (I);
removing any protecting groups; and/or
forming a pharmaceutically acceptable salt.

The process is conveniently carried out in the presence of a base such as DiPEA or TEA and one or more activating agents such as EDCI, 2-pyridinol-1-oxide, or T3P. The reaction is conveniently carried out in an organic solvent such as DMF or DCM at a temperature, for example, in the range from 20° C. to 100° C., in particular at ambient temperature (25° C.).

Compounds of formula (II) may be prepared by reaction of a compound of formula (IV),

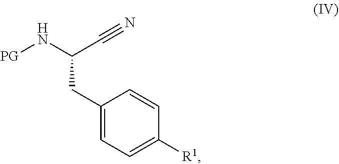

wherein PG represents a protecting group (e.g. tert-butoxycarbonyl), with a suitable reagent to remove the protecting group PG. An example of a suitable reagent is formic acid.

Compounds of formula (IV) may be prepared by reacting a compound of formula (V),

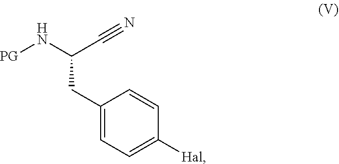

wherein PG represents a protecting group (e.g., tert-butoxycarbonyl) and Hal represents a halogen (e.g. I or Br), with a compound of formula (VI) or an ester thereof,

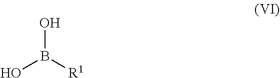

wherein R¹ is as defined in formula (I), in the presence of a catalyst such as Pd(dppf)Cl₂.DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C.

Compounds of formula (V) may be prepared from a compound of formula (VII),

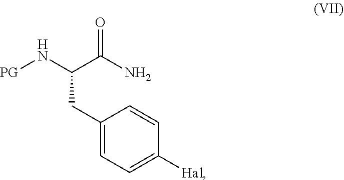

in which PG represents a protecting group (e.g. tert-butoxycarbonyl) and Hal represents a halogen (e.g., I or Br), using standard literature procedures for the dehydration of an amide, for example with Burgess reagent, or with a reagent such as T3P with or without a base such as DiPEA, in a solvent such as DCM or DMF at a temperature in the range from −20° C. to 100° C., for example at 0° C.

Compounds of formula (VII) may be prepared by reacting a compound of formula (VIII),

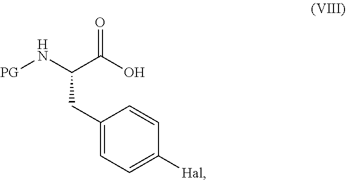

in which PG represents a protecting group (e.g. tert-butoxycarbonyl) and Hal represents a halogen (e.g., I or Br), with an aqueous ammonia solution, using standard literature procedures for the formation of an amide, for example, in the presence of a base such as N-ethyl-morpholine or DiPEA and an activating agent such as TBTU or T3P. The reaction is conveniently carried out in an organic solvent such as DMF, at a temperature in the range from −20° C. to 100° C., for example at 0° C.

Compounds of formula (VIII) are either commercially available, are known in the literature (e.g., from Tetrahedron: Asymmetry, 1998, 9, 503, incorporated by reference herein in its entirety for all purposes) or may be prepared using known techniques.

There is further provided a process for the preparation of a compound of formula (I), or a pharmaceutically acceptable salt thereof, as defined above which comprises reacting a compound of formula (IX),

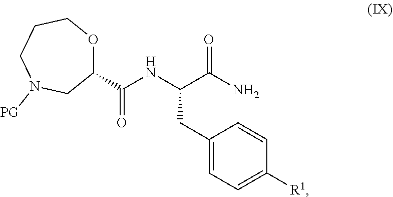

wherein R¹ is as defined above and PG represents a protecting group (e.g. tert-butoxycarbonyl), using standard literature procedures for the dehydration of an amide, for example with Burgess reagent or with a reagent such as T3P with or without a base such as DiPEA, in a solvent such as DCM or DMF at a temperature in the range from −20° C. to 100° C., for example at 25° C., and thereafter reacting with a suitable reagent to remove the protecting group PG. An example of a suitable reagent is formic acid.

A compound of formula (IX) may be prepared by reacting a compound of formula (X), wherein PG represents a protecting group (e.g. tert-butoxycarbonyl),

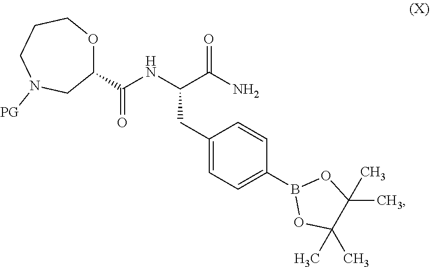

with a halide of formula (XI), wherein R¹ is defined as in formula (I),

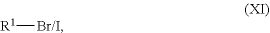

in the presence of a catalyst such as bis[bis(1,2-diphenylphosphino)ethane]palladium(0), or Pd(dppf)Cl₂ DCM, and a base such as potassium carbonate or sodium carbonate. The reaction is conveniently carried out in a solvent such as dioxane/water mixture or ACN/water mixture at a temperature, for example, in the range from 20° C. to 100° C., particularly at 80° C.

A compound of formula (X) may be prepared by reacting a compound of formula (XII), wherein PG represents a protecting group (e.g. tert-butoxycarbonyl),

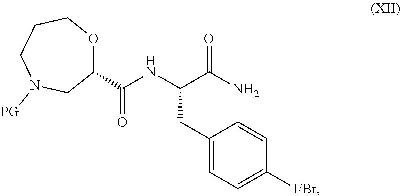

with B₂Pin₂ in the presence of a suitable catalyst such as Pd(dppf)Cl₂.DCM and with or without 1,1′-bis(diphenylphosphino)ferrocene or 1,1-bis(di-tert-butylphosphino)ferrocene palladium dichloride, with a suitable salt such as potassium acetate, in a solvent such as DMSO at a temperature in the range 60° C. to 100° C., for example at 85° C.

A compound of formula (XII) may be prepared by reacting a compound of formula (XIII),

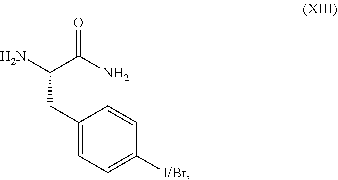

with a compound of formula (III),

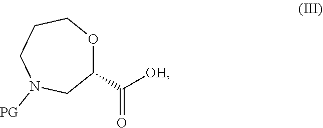

wherein PG represents a protecting group (e.g. tert-butoxycarbonyl) in the presence of a base such as DiPEA or TEA and an activating agent such as EDCI, 2-pyridinol-1-oxide, or T3P. The reaction is conveniently carried out in an organic solvent such as DMF or DCM at a temperature, for example, in the range from 20° C. to 100° C., in particular at ambient temperature (25° C.).

Compounds of formula (XIII) may be prepared by reacting a compound of formula (XIV),

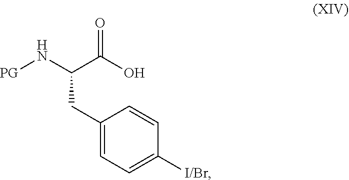

in which PG is as defined in formula (VII), with an aqueous ammonia solution, using standard literature procedures for the formation of an amide, for example, in the presence of a base such as N-ethyl-morpholine or DiPEA and an activating agent such as a “uronium” reagent (for example TBTU), or T3P. The reaction is conveniently carried out in an organic solvent such as DMF, at a temperature in the range from −20° C. to 100° C., for example at 0° C.

A compound of formula (IX) may be prepared by reacting a compound of formula (XII) wherein PG represents a protecting group (e.g. tert-butoxycarbonyl), with a compound of formula (VI) or a boronate ester thereof, in the presence of a catalyst such as bis[bis(1,2-diphenylphosphino)ethane]palladium(0) or Pd(dppf)Cl₂.DCM and a base such as potassium carbonate or sodium carbonate. The reaction is conveniently carried out in a solvent such as dioxane/water or ACN/water mixture at a temperature, for example, in the range from 20° C. to 100° C., particularly at 80° C.

There is further provided a process for the preparation of a compound of formula (I), or a pharmaceutically acceptable salt thereof, as defined above which comprises reacting a compound of formula (XV),

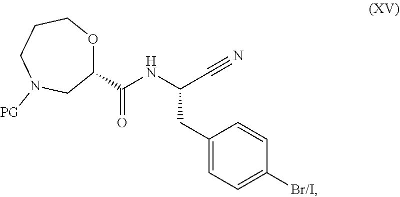

wherein PG represents a protecting group (e.g. tert-butoxycarbonyl), with a compound of formula (VI) or an ester thereof, wherein R¹ is as defined in formula (I), in the presence of a catalyst such as Pd(dppf)Cl₂.DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C., and thereafter reacting with a suitable reagent to remove the protecting group PG. An example of a suitable reagent is formic acid.

Compounds of formula (XV) may be prepared from compounds of formula (XII) using standard procedures for the dehydration of an amide, for example with Burgess reagent or a reagent such as TBTU or T3P with or without a base such as DiPEA, in a solvent such as DCM or DMF at a temperature in the range from −20° C. to 100° C., for example at 25° C.

There is further provided a process for the preparation of a compound of formula (I) or a pharmaceutically acceptable salt thereof as defined above which comprises reacting a compound of formula (XVI),

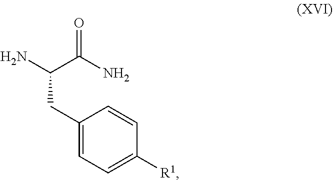

wherein R¹ is as defined in formula (I), with a compound of formula (III), conveniently carried out in the presence of a base such as DiPEA or TEA and one or more activating agents such as EDCI, 2-pyridinol-1-oxide, or T3P, followed by a dehydrating reagent such as T3P. The reaction is conveniently carried out in an organic solvent such as DMF or DCM at a temperature, for example, in the range from 20° C. to 100° C., in particular at ambient temperature (25° C.).

Compounds of formula (XVI) can be prepared from reacting compounds of formula (VII) with compounds of formula (VI) or an ester thereof, wherein R¹ is as defined in formula (I), in the presence of a catalyst such as Pd(dppf)Cl₂.DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from 20° C. to 100° C., particularly at 75° C., followed by deprotection of PG.

A compound of formula (III),

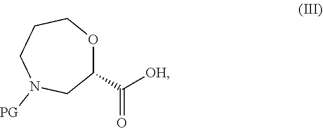

wherein PG represents a protecting group (e.g. tert-butoxycarbonyl) is either commercially available, or may be prepared from a compound of formula (XVII),

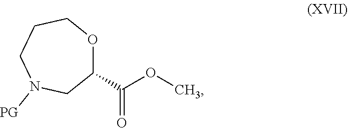

using literature procedures for mild ester hydrolysis (e.g. from Tetr. Lett., 2007, 48, 2497, incorporated by reference herein in its entirety for all purposes), for example with LiBr and a base such as TEA, in a solvent such as ACN/water mixture, for example at 25° C.

A compound of formula (XVII), wherein PG represents a protecting group (e.g. tert-butoxycarbonyl), may be prepared from a compound of formula (XVIII),

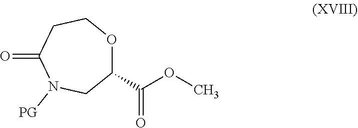

using a reducing agent, for example BH₃-DMS, in a solvent such as THF, at a temperature in the range from 0 to 40° C., for example at 25° C.

A compound of formula (XVIII), where PG represents a protecting group (e.g. tert-butoxycarbonyl), may be prepared from a compound of formula (XIX), using a biocatalytic transformation for chemoselective lactam formation, e.g., using a lipase such as Novozym 435, in a solvent such as an ether, e.g., dioxane, at a temperature in the range from 0 to 80° C., for example at 55° C., followed by conditions for introduction of the protecting group PG.

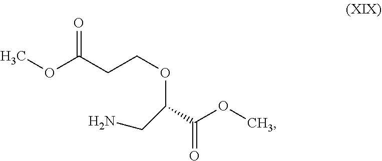

A compound of formula (XIX) may be prepared from a compound of formula (XX),

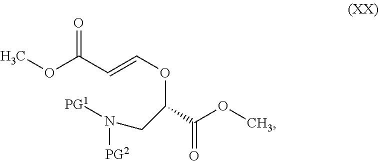

wherein PG¹ and PG² are protecting groups (e.g., benzyl), using conditions for hydrogenation, for example using H₂ (g), and a reagent such as palladium dihydroxide on carbon, in a solvent such as methanol or dioxane, under a pressure of for example 10 bar, at a temperature in the range from 25 to 80° C., for example at 40° C.

A compound of formula (XX), wherein PG¹ and PG² are protecting groups (e.g., benzyl), may be prepared from a compound of formula (XXI),

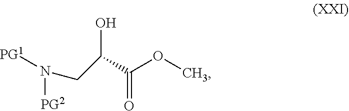

wherein PG¹ and PG² are protecting groups (e.g. benzyl), using conditions for Oxa-Michael reaction, reacting with methyl propynoate, in presence of a base such as 4-methylmorpholine, in a solvent such as toluene, at a temperature in the range from 0 to 100° C., for example at 25° C.

A compound of formula (XXI), w wherein PG¹ and PG² are protecting groups (e.g. benzyl), may be prepared from reacting a diprotected benzyl amine (e.g., dibenzylamine) with (S)-methyl oxirane-2-carboxylate, in a solvent such as ethanol, at a temperature in the range from 0 to 78° C., for example at 70° C.

Alternatively, a compound of formula (III),

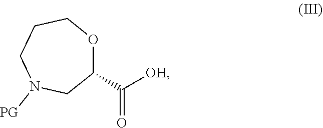

wherein PG represents a protecting group (e.g. tert-butoxycarbonyl) may be prepared from oxidation of a compound of formula (XXII),

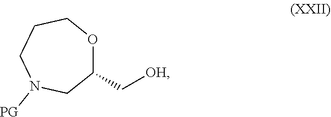

for example, using reagents such as TEMPO, and sodium hypochlorite, optionally in presence of a salt such as sodium bromide, in a solvent such as DCM/water, and in presence of a buffer such as NaHCO₃, and a phase transfer catalyst such as tetrabutylammonium bisulphate, at a temperature in the range from 0 to 100° C., e.g., at 25° C.

A compound of formula (XXII), wherein PG represents a protecting group (e.g., tert-butoxycarbonyl) may be prepared from a compound of formula (XXIII),

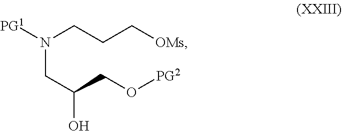

wherein PG¹ and PG² are protecting groups (e.g. benzyl), reacting with a base such as sodium hydride, in a solvent such as THF, at a temperature in the range from 0 to 60° C., e.g., 25° C., followed by interconversion of protecting groups PG, PG¹ and PG², as defined in formula (XXII) and (XXIII).

A compound of formula (XXIII), wherein PG¹ and PG² are protecting groups (e.g., benzyl), may be prepared from reacting protected 3-aminopropanol (e.g. N-benzyl-3-aminopropanol) with (S)-2-((benzyloxy)methyl)oxirane, in a solvent such as ethanol or propanol, at a temperature in the range from 0 to 70° C., for example at 40° C., followed by reacting the crude product with methanesulfonyl chloride, in presence of a base such as DiPEA, in a solvent such as DCM, at a temperature in the range from −10 to 25° C., e.g., −5° C.

Compounds of formula (VI) or an ester thereof, (VIII), (XI) and (XIV) are either commercially available, are known in the literature or may be prepared using known techniques.

It will be appreciated by those skilled in the art that in the processes of the present disclosure certain functional groups such as hydroxyl or amino groups in the reagents may need to be protected by protecting groups. Thus, the preparation of the compounds of formula (I) may involve, at an appropriate stage, the removal of one or more protecting groups.

The skilled person will recognise that at any stage of the preparation of the compounds of formula (I), mixtures of isomers (e.g., racemates) of compounds corresponding to any of formulae (II)-(V), (VII)-(X) and (XXII)-(XVI) may be utilized. At any stage of the preparation, a single stereoisomer may be obtained by isolating it from a mixture of isomers (e.g., a racemate) using, for example, chiral chromatographic separation.

The protection and deprotection of functional groups is described in ‘Protective Groups in Organic Synthesis’, 4^{th} Ed, T. W. Greene and P. G. M. Wuts, Wiley (2006) and ‘Protecting Groups’, 3^{rd} Ed P. J. Kocienski, Georg Thieme Verlag (2005), incorporated by reference herein in its entirety for all purposes.

As provided throughout, according to the methods provided herein, a compound of formula (I) can be administered as a pharmaceutically acceptable salt. A pharmaceutically acceptable salt of a compound of formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of formula (I).

Where the compound of formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of formula (I) is sufficiently basic, pharmaceutically acceptable salts include, but are not limited to, inorganic or organic acid addition salts.

There may be more than one cation or anion depending on the number of charged functions and the valency of the cations or anions.

For reviews on suitable salts, and pharmaceutically acceptable salts amenable for use herein, see Berge et al., J. Pharm. Sci., 1977, 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes.

The compounds of formula (I) may form mixtures of its salt and co-crystal forms. It is also to be understood that the methods provided herein can employ such salt/co-crystal mixtures of the compound of formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that certain compounds of formula (I) may exist in solvated form, e.g., hydrates, including solvates of a pharmaceutically acceptable salt of a compound of formula (I).

In one embodiment, certain compounds of formula (I) may exist as racemates and racemic mixtures, single enantiomers, individual diastereomers and diastereomeric mixtures.

It is to be understood that the present disclosure encompasses all such isomeric forms. Certain compounds of formula (I) may also contain linkages (e.g., carbon-carbon bonds, carbon-nitrogen bonds such as amide bonds) wherein bond rotation is restricted about that particular linkage, e.g. restriction resulting from the presence of a ring bond or double bond. Accordingly, it is to be understood that the methods provided herein can employ such isomers. Certain compound of formula (I) may also contain multiple tautomeric forms. It is to be understood that the present disclosure encompasses all such tautomeric forms. Stereoisomers may be separated using conventional techniques, e.g. chromatography or fractional crystallization, or the stereoisomers may be made by stereoselective synthesis.

In a further embodiment, the compounds of formula (I) encompass any isotopically-labeled (or “radio-labelled”) derivatives of a compound of formula (I). Such a derivative is a derivative of a compound of formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of formula (I) is provided where one or more hydrogen atoms are replaced by one or more deuterium atoms; and the deuterated compound is used in one of the methods provided herein for treating IBD.

In a further embodiment, the compounds of formula (I) may be administered in the form of a prodrug which is broken down in the human or animal body to give a compound of the formula (I). Examples of prodrugs include in vivo hydrolysable esters of a compound of the formula (I).

An in vivo hydrolysable (or cleavable) ester of a compound of the formula (I) that contains a carboxy or a hydroxy group is, for example, a pharmaceutically acceptable ester which is hydrolyzed in the human or animal body to produce the parent acid or alcohol. For examples of ester prodrugs derivatives, see: Curr. Drug. Metab. 2003, 4, 461, incorporated by reference herein in its entirety for all purposes.

Various other forms of prodrugs are known in the art, and can be used in the methods provided herein. For examples of prodrug derivatives, see: Nature Reviews Drug Discovery 2008, 7, 255, the disclosure of which is incorporated by reference herein in its entirety for all purposes.

### EXAMPLES

The present invention is further illustrated by reference to the following Examples. However, it should be noted that these Examples, like the embodiments described above, are illustrative and are not to be construed as restricting the scope of the invention in any way.

### Example 1—INS1007 Treatment of DSS-induced IBD

The efficacy of INS1007 in the treatment of DSS-induced colitis in C57BL/6 mice was evaluated. Example 1A evaluated the effectiveness of INS1007 dosed at 0.1 mg/kg, 1.0 mg/kg and 10 mg/kg in the treatment of DSS-induced colitis model. Example 1B evaluated the effectiveness of INS1007 dosed at 0.3 mg/kg, 1.0 mg/kg, 3.0 mg and 10 mg/kg in the treatment of DSS-induced colitis model. In Examples 1A and 1B, vehicle (negative control) and an anti-p40 mAb antibody (positive control) were also evaluated.

### Example 1A

Mice: Seventy-nine (79) male C57BL/6 mice with an average starting body weight (SEM) of 23.32 g (+1.53 g) were obtained from Charles River Laboratories (Wilmington, Mass.). Animals were acclimatized prior to the study. During this period, the animals were observed daily in order to reject any that presented in poor condition. Animals were fed with 5053 sterile rodent diet and water was provided ad libitum.

INS1007 Solutions: INS1007 was diluted with 0.5% hydroxypropylmethylcellulose in citrate buffer w/0.1% Tween 80 (pH 3.0). Solutions were stored at 4° C. INS1007 solutions were discarded after 3 days and fresh solutions were prepared.

Anti-p40 mAb solution: Anti-mouse IL-12 p40 (Clone: C17.8) obtained from BioXCell (Cat #: BE0051) was diluted with phosphate buffered saline. Solutions were stored at 4° C. Anti-mouse IL-12 p40 solutions were discarded after 7 days and fresh solutions were prepared.

DSS Induction Solution: Dextran sulfate sodium salt obtained from MP Biomedical (Cat #02160110) was diluted with water. DSS solutions were stored at room temperature.

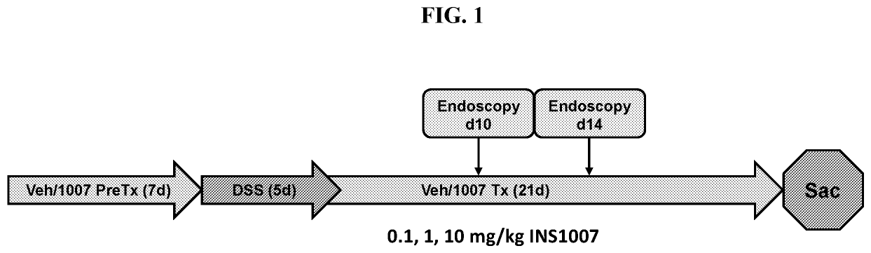

Methods: The study protocol is summarized in FIG. 1ⁱ and Table 1. Mice were sorted into eight groups as shown in Table 1. Groups 1-4 were negative controls; groups 5-7 were treated with INS 1007 and Group 8 was treated with Anti-p40 mAb. ⁱ The dosing protocol for the Anti-p40 antibody is not shown in FIG. 1.

The study included three phases: a pre-treatment period (Veh/1007 Pretx in FIG. 1); DSS-induction period (DSS in FIG. 1) and a treatment period (Veh/1007 in FIG. 1). Colitis was induced in Groups 3-8 by exposure to 3% DSS Induction Solution from Day 0 to Day 5. Animals in Groups 2 and 4-7 were dosed via oral gavage (PO) twice daily (BID) from Days −7 to 20 with INS1007 Solution or vehicle, as indicated in Table 1. Animals in Group 8 were dosed via intraperitoneal (IP) injection once every third day (Q3D) from Day 0 to 18 with Anti-p40 mAb solution.

TABLE 1
Study Protocol

| | Number | DSS | | | | |
|---|---|---|---|---|---|---|
| | of | Day | | | | Dose |
| Group | Animals | 0-5 | Treatment | Dose | Route | Schedule |
| 1 | 4 | — | — | — | — | — |
| 2 | 4 | — | Vehicle | — | PO | BID |
| | | | | | 10 ml/kg | Day (-7) to 20 |
| 3 | 8 | 3% | — | — | — | — |
| 4 | 15 | 3% | Vehicle | — | PO | BID |
| | | | | | 10 ml/kg | Day (-7) to 20 |
| 5 | 12 | 3% | IN51007 | 0.1 | PO | BID |
| | | | | mg/kg | 10 ml/kg | Day (-7) to 20 |
| 6 | 12 | 3% | INS1007 | 1 | PO | BID |
| | | | | mg/kg | 10 ml/kg | Day (-7) to 20 |
| 7 | 12 | 3% | INS1007 | 10 | PO | BID |
| | | | | mg/kg | 10 ml/kg | Day (-7) to 20 |
| 8 | 12 | 3% | Anti-p40 | 10 | IP | Q3D |
| | | | mAb | mg/kg | 5 ml/kg | Day 0, 3, 6, 9, |
| | | | | | | 12, 15, 18 |

All animals were weighed daily and assessed visually for the presence of diarrhea and/or bloody stool at the time of dosing. Each mouse, under isoflurane anesthesia, underwent video endoscopy on Days 10, 14, & 21 using a small animal endoscope (Karl Storz Endoskope, Germany), to assess colitis severity. Colitis severity was scored using a 0-4 scale (0=normal; 1=loss of vascularity; 2=loss of vascularity and friability; 3=friability and erosions; 4=ulcerations and bleeding). Stool consistency was scored during endoscopy using the parameters defined in Table 2. Following endoscopy on day 21, all animals from each treatment group were sacrificed.

TABLE 2
Stool Consistency

| Score | Description: |
|---|---|
| 0 | Normal, well-formed pellet |
| 1 | Loose stool, soft, staying in shape |
| 2 | Loose stool, abnormal form with excess moisture |
| 3 | Watery or diarrhea |
| 4 | Bloody diarrhea |

Following euthanasia, bone marrow (from both femurs and tibias) were collected, lysed, and then snap frozen. The frozen tissue was stored at −80° C.

Results:

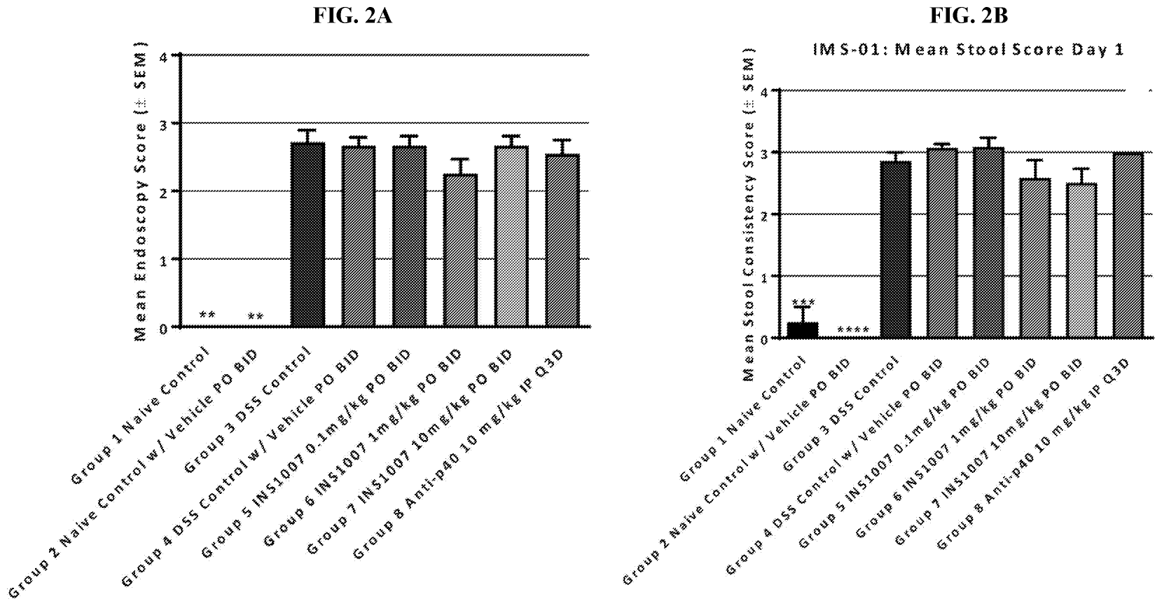

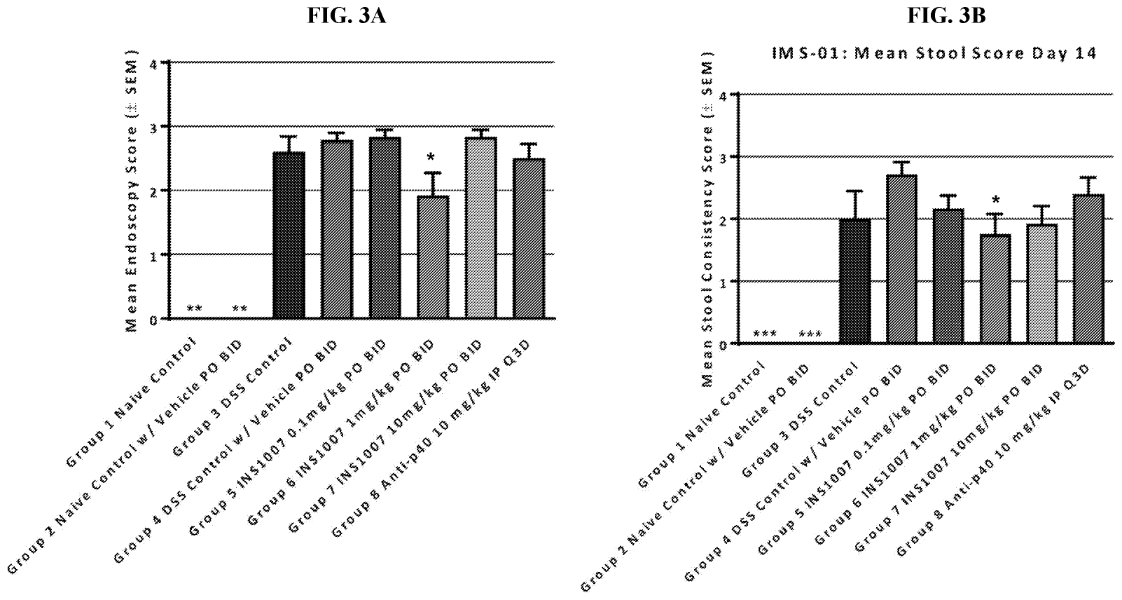

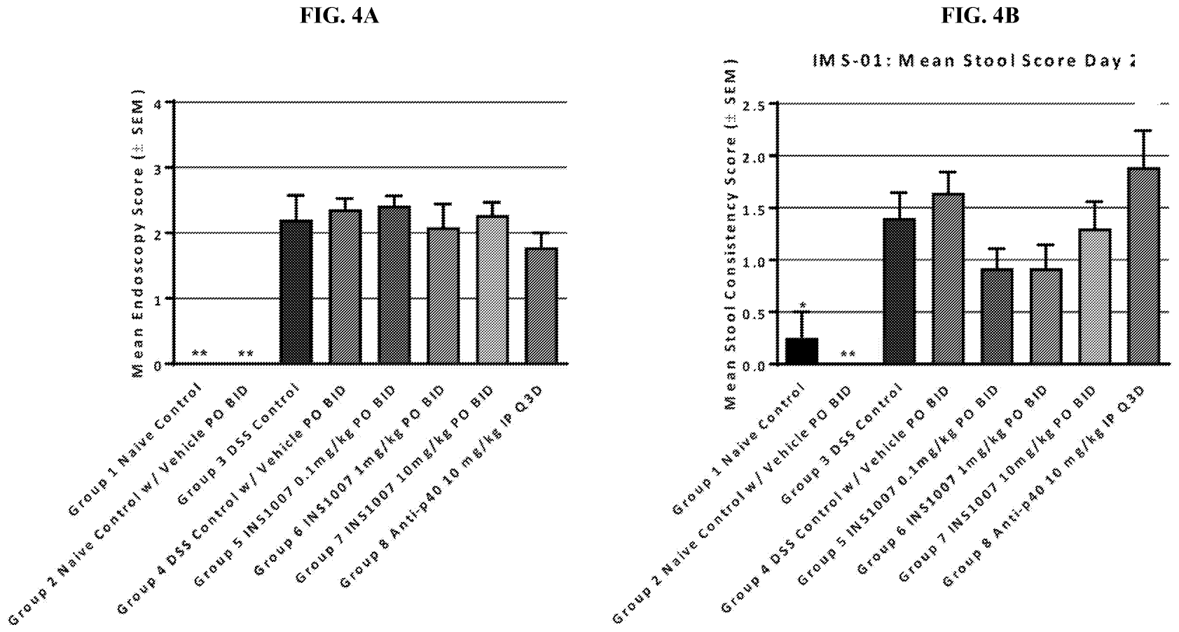

Mean Endoscopy Score: Animals underwent video endoscopy on Days 10, 14, and 21 to assess the severity of colitis in each treatment group. Mean endoscopy scores for Days 10, 14 and 21 are presented in FIGS. 2a, 3a and 4a, respectively.

On all days of evaluation, colitis was observed in all groups that received DSS (Groups 3-8). There was no colitis in the naïve control groups (i.e., colitis not induced with DSS) as evidenced by scores of 0 throughout this study. This effect was statistically significant compared to the DSS-Vehicle Control Group (p<0.01 for both naïve groups on all three days of evaluation) The DSS-Vehicle controls reached mean scores of 2.67, 2.79, and 2.36 on Days 10, 14, and 21, respectively. Treatment with INS1007 at the 1 mg/kg concentration decreased the mean endoscopy scores on each evaluation day (Day 10=2.25; Day 14=1.92, Day 21=2.08). Anti-p40 dosed Q3D from Day 0 to 21 also produced reduction in mean endoscopy scores.

Stool consistency: Animals underwent video endoscopy on Days 10, 14 and 21 to assess the stool consistency in each treatment group. Mean stool consistency scores for Days 10, 14 and 21 are presented in FIGS. 2b, 3b and 4b, respectively. Both naïve control groups had a very low stool consistency score, which was significantly lower than DSS-induced animals dosed with Vehicle at all three evaluation time-points (p<0.05-p<0.0001). Treatment with INS1007 at all three concentrations led to modest decreases in stool scores on all three days of evaluation, but particularly on Days 14 and 21. Treatment with Anti-p40 exhibited little effect on stool consistency scores.

Weight Loss: Weight loss, calculated as a percentage of the starting weight on Day 0 was observed in all treatment groups. The DSS-vehicle control group reached peak weight loss on Day 11 (−18.46%), then slowly recovered weight up until sacrifice on Day 21. Treatment with INS1007 and Anti-p40 (10 mg/kg) every three days modestly attenuated weight loss. The naïve control group and the naïve control group dosed with Vehicle exhibited weight gain throughout the duration of the study (with the vehicle-dosed naïve group exhibiting slightly reduced weight gain than fully naïve animals).

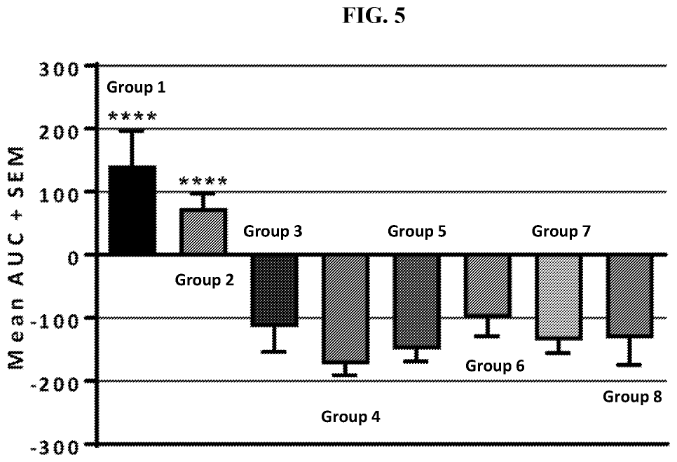

To assess the statistical significance of overall differences in weight change under various test treatments following DSS administration, the area under the curve (AUC) for Days 0 to 21 was calculated. The mean AUC for each group is shown in the FIG. 5. The naïve control groups had significantly increased weight gain compared to the DSS-vehicle control group (Group 4; p<0.0001), but no other comparisons were statistically significant.

Bone Marrow Neutrophil Activity: The ability of INS1007 to reduce neutrophil activation in DSS-induced colitis in C57BL/6 mice was evaluated. The neutrophil activity of bone marrow extracts taken from the euthanized mice of Example 1A was observed.

All animals from Example 1A had both femurs and tibias removed and bone marrow was aspirated out with ice-cold using Roswell Park Memorial Institute (RPMI) buffer. Cell pellets were collected after red blood cell lysis and wash with PBS. Ice-cold PBS-Triton X-100 lysis buffer was added to the cell pellet to obtain cell lysate after maximum speed centrifugation. Cell lysate were stored at 80° C. until enzymatic assay test.

For neutrophil elastase (NE) activity assay, bone marrow cell lysates were added to 96-well black plate and incubated for 15 minutes in the presence of DMSO or NE inhibitor (Abcam, Cat #ab142154). A serial dilution of natural mouse elastase protein (Abcam, Cat #ab95133) was used for establishing the standard curve. The NE substrate (Methoxysuccinyl-ala-ala-pro-val-AMC; Sigma, Cat #M9771) was added for both samples and NE inhibitor controls. The resulting fluorescence was measured at 350 nm excitation and 450 nm emission in a kinetic mode by reading every 5 minutes for up to 3 hours at 37° C. The fluorescence was read using a BioTek Synergy plate reader. The reaction rate was calculated with the initial slopes of the linear range and a standard curve for the natural mouse elastase amounts were plotted against their slopes. The net NE activity (in the unit of natural mouse elastase amount) by subtracting the NE inhibitor control wells from non-inhibited well were calculated based on the standard curve.

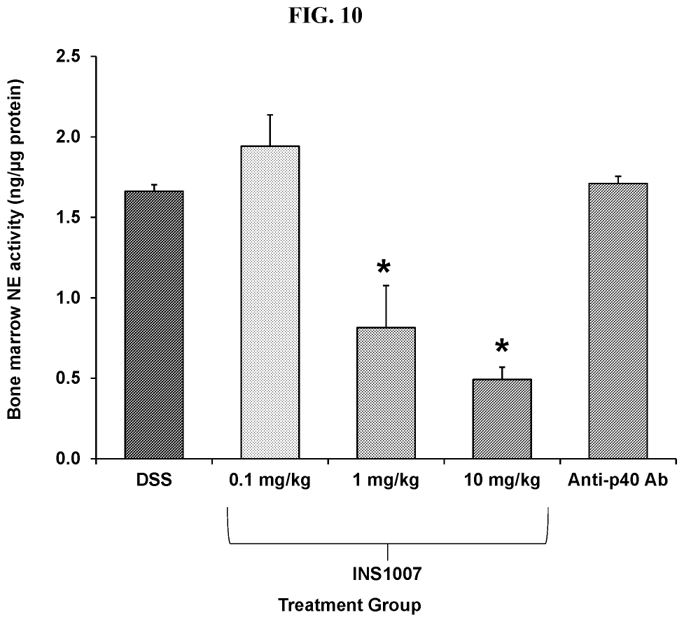

FIG. 10 shows the bone marrow neutrophil activity for samples taken from the euthanized mice of Example 1A. The neutrophil activity for the 1 mg/kg and 10 mg/kg INS1007 treated groups were significantly reduced compared to vehicle treated control group (Group 2).

### Example 1B

Mice: Ninety-five (95) male C57BL/6 mice with an average starting body weight (SEM) of 20.6 g (+0.16 g) were obtained from Charles River Laboratories (Wilmington, Mass.). Animals were acclimatized prior to the study. During this period, the animals were observed daily in order to reject any that presented in poor condition. Animals were fed with 5053 sterile rodent diet and water was provided ad libitum.

Solutions: The INS1007 Solution, Anti-p40 mAb solution and DSS Induction Solution were prepared and stored as described in Example 1A.

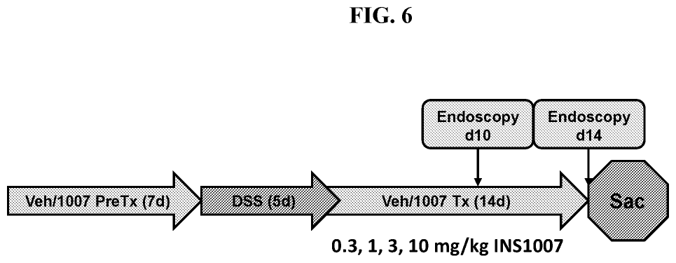

Methods: The study protocol is summarized in FIG. 6ⁱⁱ and Table 3. Mice were sorted into eight groups as shown in Table 1. Groups 1-2 were negative controls; groups 3-6 were treated with INS 1007 and Group 7 was treated with Anti-p40 mAb. ⁱⁱ The dosing protocol for the anti-p40 antibody is not shown in FIG. 6.

The study included three phases: a pre-treatment period (Veh/1007 Pretx in FIG. 6); DSS-induction period (DSS in FIG. 6) and a treatment period (Veh/1007 Tx in FIG. 6). Colitis was induced in Groups 3-7 by exposure to 3% DSS Induction Solution from Day 0 to Day 5. Animals in Groups 1-6 were dosed via oral gavage (PO) twice daily (BID) from Days −7 to 13 with INS1007 Solution or vehicle, as indicated in Table 1. Animals in Group 7 were dosed via intraperitoneal (IP) injection once every third day (Q3D) from Day 0 to 12 with Anti-p40 mAb solution.

TABLE 3
Study protocol

| | Number | DSS | | | | |
|---|---|---|---|---|---|---|
| | of | Day | | | | Dose |
| Group | Animals | 0-5 | Treatment | Dose | Route | Schedule |
| 1 | 5 | — | Vehicle | — | PO | BID |
| | | | | | 10 ml/kg | Day (-7) to 13 |
| | | | | | PO | BID |
| 2 | 15 | 3% | Vehicle | — | 10 ml/kg | Day (-7) to 13 |
| 3 | 15 | 3% | INS1007 | 0.3 mg/kg | PO | BID |
| | | | | | 10 ml/kg | Day (-7) to 13 |
| 4 | 15 | 3% | INS1007 | 1 mg/kg | PO | BID |
| | | | | | 10 ml/kg | Day (-7) to 13 |
| 5 | 15 | 3% | INS1007 | | PO | BID |
| | | | | 3mg/kg | 10 ml/kg | Day (-7) to 13 |
| 6 | 15 | 3% | INS1007 | 10 mg/kg | PO | BID |
| | | | | | 10 ml/kg | Day (-7) to 13 |
| 7 | 15 | 3% | Anti-p40 | 10 mg/kg | IP | Q3D |
| | | | mAb | | 5 ml/kg | Day 0, 3, 6, |
| | | | | | | 9, & 12 |

All animals were weighed daily and assessed visually for the presence of diarrhea and/or bloody stool at the time of dosing. Each mouse, under isoflurane anesthesia, underwent video endoscopy on Days 10 and 14, & 21 using a small animal endoscope (Karl Storz Endoskope, Germany), to assess colitis severity. Colitis severity was scored using the scale described in Example 1A. Stool consistency was scored during endoscopy using the parameters defined in Table 2. Following endoscopy on day 14, all animals from each treatment group were sacrificed. Following euthanasia, bone marrow (from both femurs and tibias) were collected, lysed, and then snap frozen. The frozen tissue was stored at −80° C.

Results:

Mean Endoscopy and Mean Stool Consistency Score:

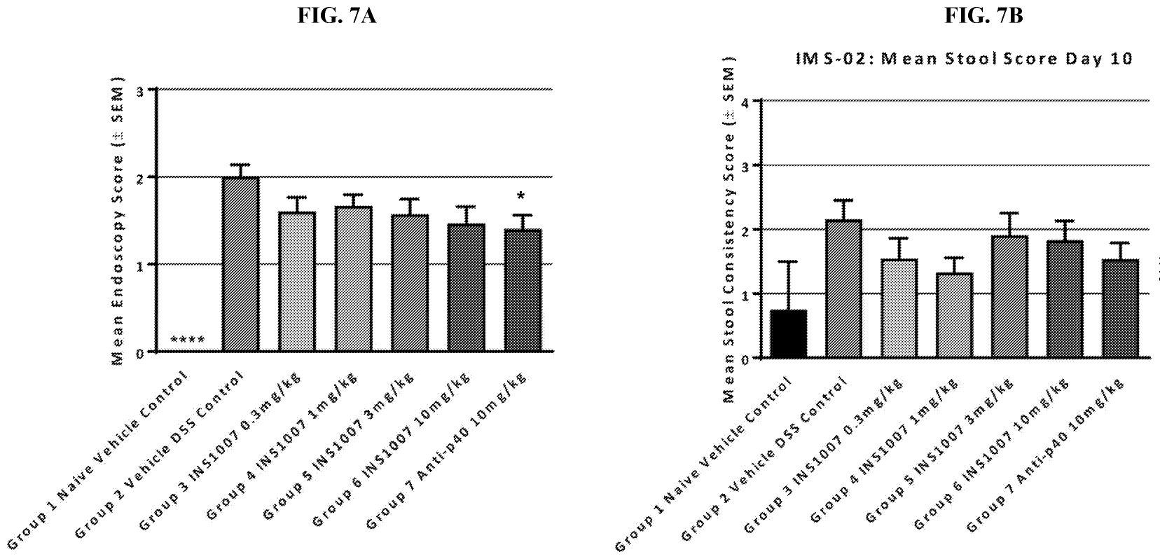

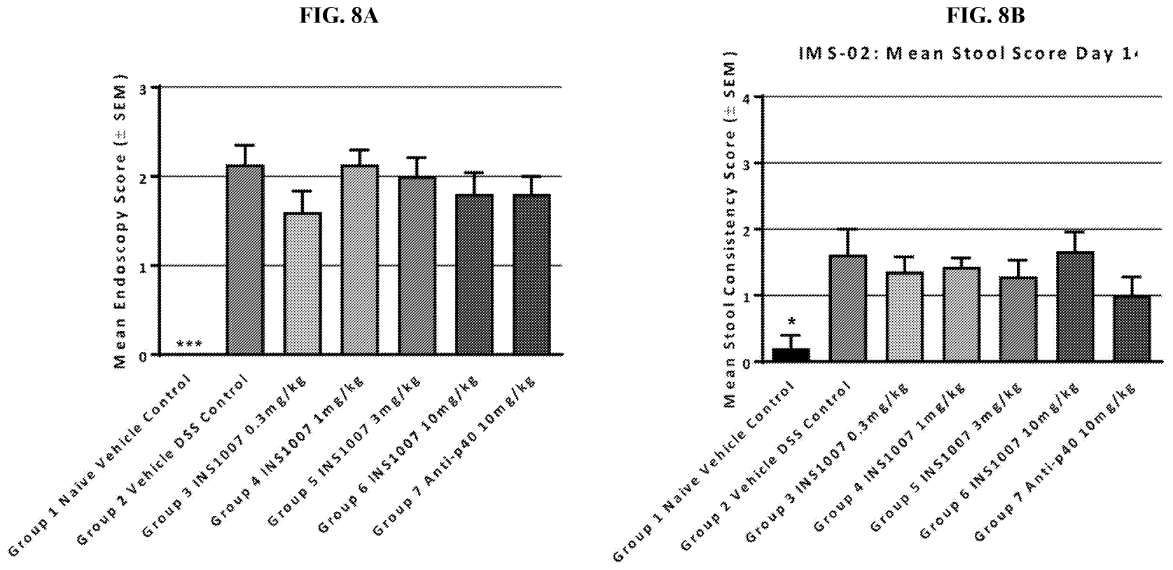

Animals underwent endoscopy on Days 10 and 14, in order to assess colonic inflammation. Colitis was scored visually on a five-point scale using the method described in Example 1A. Stool consistency was scored visually on a five-point scale using the method described in Example 1A. The mean endoscopy scores for Days 10 and 14 are shown in FIGS. 7A and 8A, respectively. Mean stool consistency scores for Days 10 and 14 are shown in FIGS. 7B and 8B, respectively.

Colitis severity and stool consistency scores for all colitis-induced groups increased at both the Day 10 (FIGS. 7A and 7B) and Day 14 (FIGS. 8A and 8B) timepoints as compared to naïve animals. Colitis severity scores were significantly reduced in naïve animals as compared to the DSS+ vehicle control group. The mean endoscopy score in the 0.3 mg/kg INS1007 group (Group 3) was reduced on Day 14 as compared to vehicle control (Group 2).

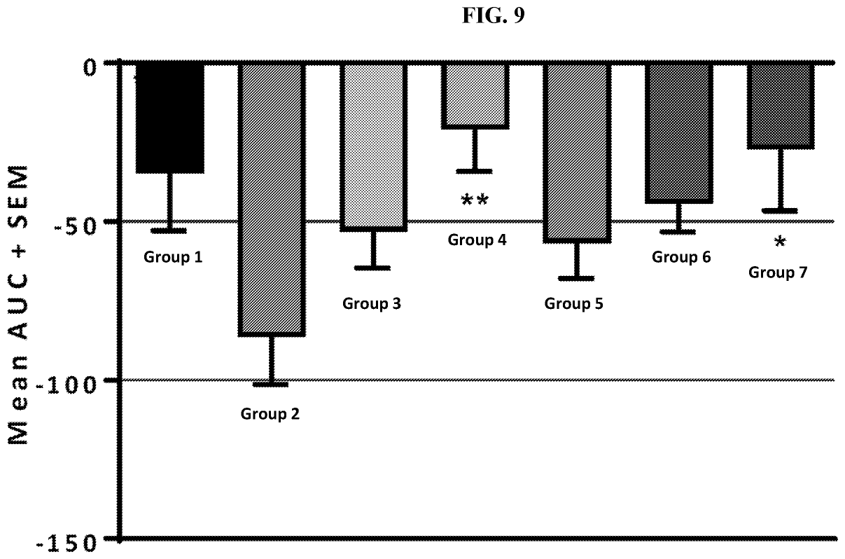

Weight Loss: Weight loss, calculated as a percentage of the starting weight on Day 0 was observed in all DSS-induced treatment groups. To determine the statistical significance of overall differences in mean percent body weight change between groups, the area under the curve (AUC) was calculated using the trapezoidal rule transformation and is shown in the FIG. 9.

No major differences in body weight change were observed prior to colitis induction (Days −7 to 0). Animals with colitis induced by DSS demonstrated body weight loss from Days 5 to 9. Body weight increased from Day 9 to Day 10 and remained steady for the remainder of the study. However, body weight did not return to baseline in animals exposed to DSS. When calculated based on Day −7, body weight loss in 1 mg/kg INS1007 (Group 4) and anti-p40 (Group 7) treated groups was significantly decreased as compared to Group 2 (3% DSS+vehicle).

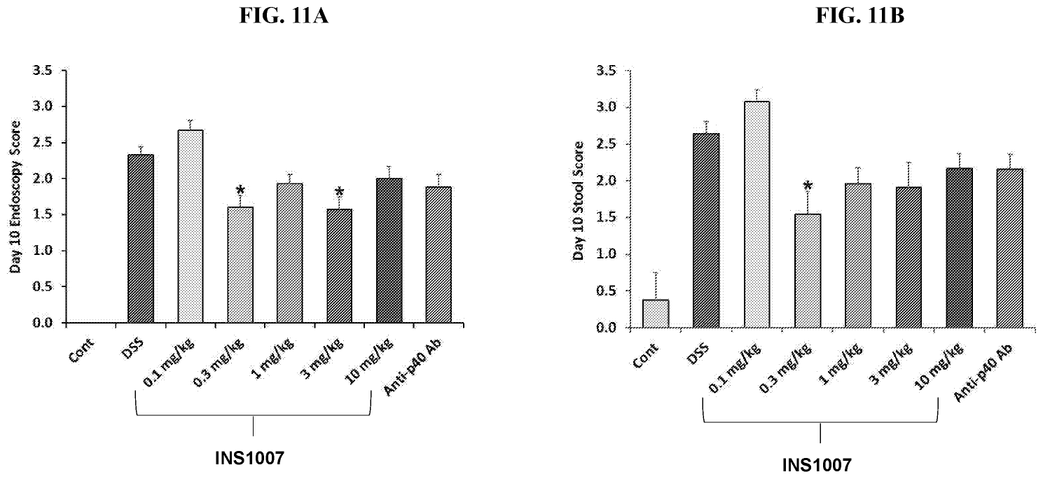

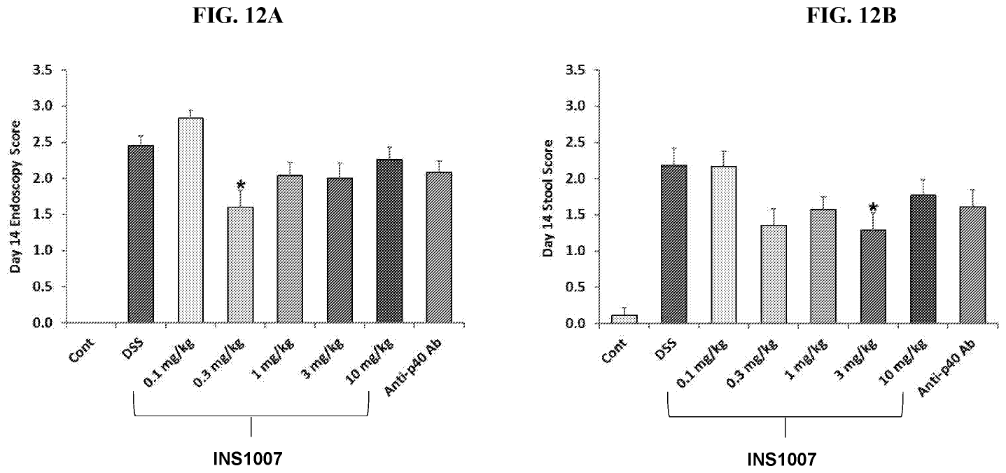

Mean Endoscopy/Stool Consistency Scores and Mean Weight Change: The mean of the endoscopy and stool consistency scores from Days 10 and 14 observed from Examples 1A and 1B was determined to provide a mean endoscopy score and mean stool consistency score for Days 10 and 14 in the DSS-induced colitis model (i.e., mean that includes the animals from Examples 1A and 1B). FIGS. 11A and 12A show the Mean Endoscopy score. FIGS. 11B and 12B show the Mean Stool Consistency score.

At Day 10, the mean endoscopy and mean stool consistency scores in the 0.3 mg/kg INS1007 group and the mean endoscopy score for in the 3 mg/kg INS group was significantly reduced as compared to vehicle control. At Day 14, the average mean endoscopy score in the 0.3 mg/kg INS1007 group and the stool consistency score in the 3 mg/kg INS group was significantly reduced as compared to vehicle control.

The percent change in body weight on Day 14 from Examples 1A and 1B were averaged to provide mean percent body weight change for Day 14 in the DSS-induced colitis model (i.e., mean that includes the animals from Examples 1A and 1B). The average mean weight change in the 0.3 mg/kg INS1007 group was significantly reduced as compared to vehicle control. That is, at Day 14, mice treated with 0.3 mg/kg INS1007 lost significantly less weight than vehicle-treated mice.

### Example 2—INS1007 Treatment of T-Cell Induced IBD

The efficacy of INS1007 in the treatment of colitis induced by the adoptive transfer of sorted naïve T cells into RAG2^{-/-} mice (“Adoptive T-Cell Transfer Colitis Model”) was evaluated. The Adoptive T-Cell Transfer Colitis Model is an immunological animal model of colitis that provides focused and detailed examination of T cell-mediated pathological mechanisms, and generally responds well to established clinical therapies such as anti-TNFα. The model is described in detail in D. V. Ostanin, et al., T cell transfer model of chronic colitis: concepts, considerations, and tricks of the trade. Am. J. Physiol. Gastrointest. Liver Physiol. 2009; 296(2): G135-46, which is hereby incorporated by reference in its entirety for all purposes.

Example 2 evaluated the effectiveness of INS1007 dosed at 0.3 mg/kg, 1.0 mg/kg, 3.0 mg/kg and 10 mg/kg in the Adoptive T-Cell Transfer Colitis Model. Vehicle (negative control) and an anti-p40 mAb antibody (positive control) were also evaluated.

Mice: Ninety-five (95) male RAG2^{-/-} mice (n=95) with an average starting body weight (±SEM) of 21.3±0.12 g were obtained from Taconic Biosciences (Germantown, N.Y.). Animals were acclimatized prior to study commencement. During this period, the animals were observed daily in order to reject any that presented in poor condition. Animals were fed with 5053 sterile rodent diet and water was provided ad libitum.

INS1007 Solutions: INS1007 was diluted with 0.5% Hydroxypropylmethylcellulose in Citrate buffer w/0.1% Tween 80 (pH 3.0). Solutions were stored at 4° C. INS1007 solutions were discarded after 3 days and fresh solutions were prepared.

Anti-p40 mAb solution: Anti-mouse IL-12 p40 (Clone: C17.8) obtained from BioXCell (Cat #: BE0051) was diluted with phosphate buffered saline. Solutions were stored at 4° C. Anti-mouse IL-12 p40 solutions were discarded after 7 days and fresh solutions were prepared.

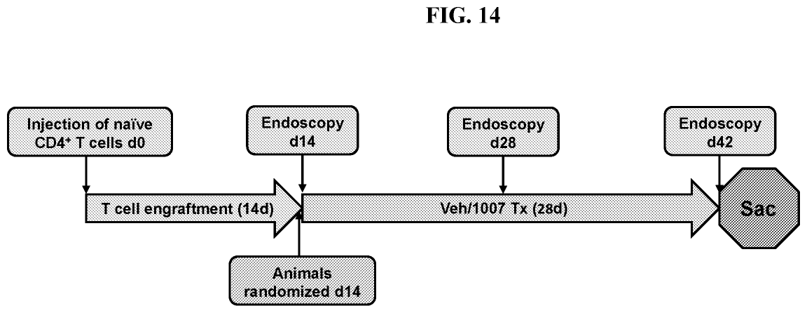

Methods: The study protocol is summarized in FIG. 14 and Table 4. Mice were sorted into seven groups as shown in Table 4. Groups 1-2 were negative controls; groups 3-6 were treated with INS 1007 and Group 7 was treated with Anti-p40 mAb.

The study included two phases: a T-cell transfer colitis-induction period (T-cell engraftment in FIG. 14) and a treatment period (Veh/1007 TX in FIG. 14). Colitis was induced on Day 0 in 90 RAG2^{-/-} mice by IP injection (200 μL) of 0.5×10⁶ CD44⁻/CD62L⁺ T-cells (in PBS) isolated and purified from C57Bl/6 recipients. Donor cells were processed as follows. Whole spleens were excised from donor C57BL/6 mice and were immediately placed ice-cold PBS. The spleens were dissociated to yield a single cell suspension and the red blood cells were lysed. Spleens were then processed for CD4+ enrichment prior to CD44-CD62L⁺ sorting using Miltenyi MACS columns.

Animals in Groups 1-6 were dosed via oral gavage (PO) twice daily (BID) from Days 14 to 41 with INS1007 Solution or vehicle, as indicated in Table 4. Animals in Group 7 were dosed via intraperitoneal (IP) injection once every third day (Q3D) from Day 14 to 39 with Anti-p40 mAb solution.

TABLE 4
Study Protocol

| | Num- | Native | | | | |
|---|---|---|---|---|---|---|
| | ber of | T-Cell | | | | Dose |
| | Ani- | Trans- | Treat- | | | |
| Group | mals | fer | ment | Dose | Route | Schedule |
| 1 | 5 | — | Vehicle | — | PO | BID |
| | | | | | 10 ml/kg | Day 14 to 41 |
| 2 | 15 | 0.5 × | Vehicle | — | PO | BID |
| | | 106 | | | 10 ml/kg | Day 14 to 41 |
| 3 | 15 | 0.5 × | INS1007 | 0.3 | PO | BID |
| | | 106 | | mg/kg | 10 ml/kg | Day 14 to 41 |
| 4 | 15 | 0.5 × | INS1007 | 1 | PO | BID |
| | | 106 | | mg/kg | 10 ml/kg | Day 14 to 41 |
| 5 | 15 | 0.5 × | INS1007 | 3 | PO | BID |
| | | 106 | | mg/kg | 10 ml/kg | Day 14 to 41 |
| 6 | 15 | 0.5 × | INS1007 | 10 | PO | BID |
| | | 106 | | mg/kg | 10 ml/kg | Day 14 to 41 |
| 7 | 15 | 0.5 × | Anti-p40 | 10 | IP | Q3D |
| | | 106 | mAb | mg/kg | 5ml/kg | Day 14, 17, 20, |
| | | | | | | 23, 27, 30, 33, |
| | | | | | | 36, & 39 |

Animals were observed daily (weight morbidity, survival, presence of diarrhea and/or bloody stool) to assess differences among treatment groups and/or possible toxicity resulting from the treatments. Animals exhibiting a weight loss greater than 30%, or those demonstrating moribundity, were euthanized; blood and bone marrow samples were collected from these animals.

Each mouse, under isoflurane anesthesia, underwent video endoscopy on Days 14, 28, and 42 using a small animal endoscope (Karl Storz Endoskope, Germany), to assess colitis severity. During each endoscopic procedure, still images and video were recorded to evaluate the extent of colitis and the response to treatment. Colitis severity and Stool consistency were scored using the methods described in Examples 1 and 2. Following endoscopy on day 42, all animals from each treatment group were sacrificed. Following euthanasia, bone marrow (from both femurs and tibias) were collected, lysed, and then snap frozen. The frozen tissue was stored at −80° C.

Results:

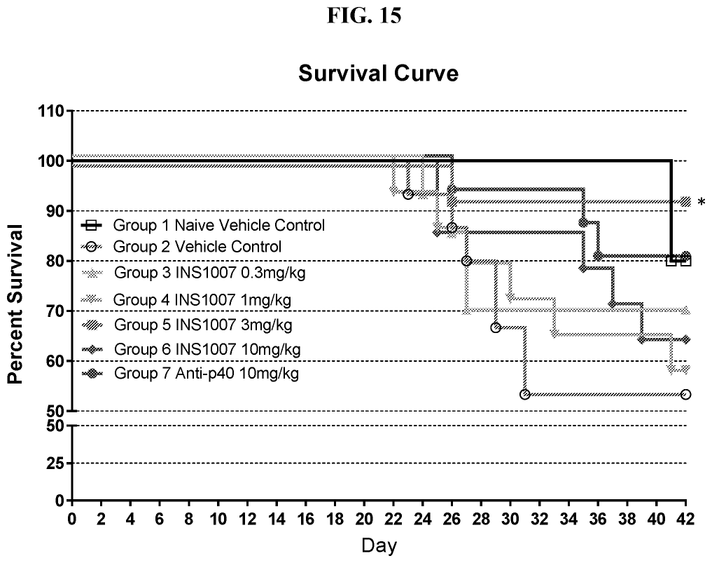

Survival: Animals were assessed daily for survival or moribundity, and a Kaplan-Meier curve showing survival over the duration of the study is shown in FIG. 15. Survival was 80% in T-cell naïve group (Group 1), 53.3% in the adoptive transfer/vehicle control group (Group 2), and varied in other treatment groups. Survival in 3 mg/kg INS1007 treatment group (Group 5) demonstrated the best survival overall (95.86%). Survival in Group 5 was significantly improved as compared to Vehicle control (Group 3).

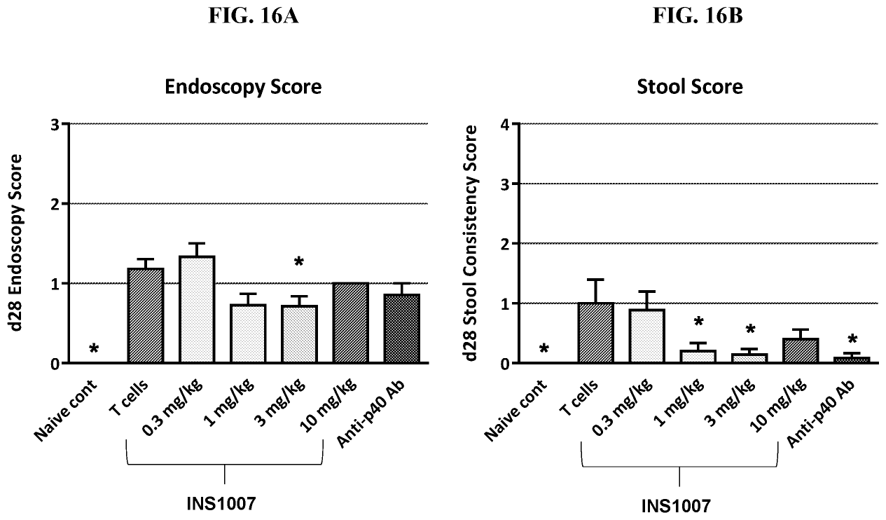

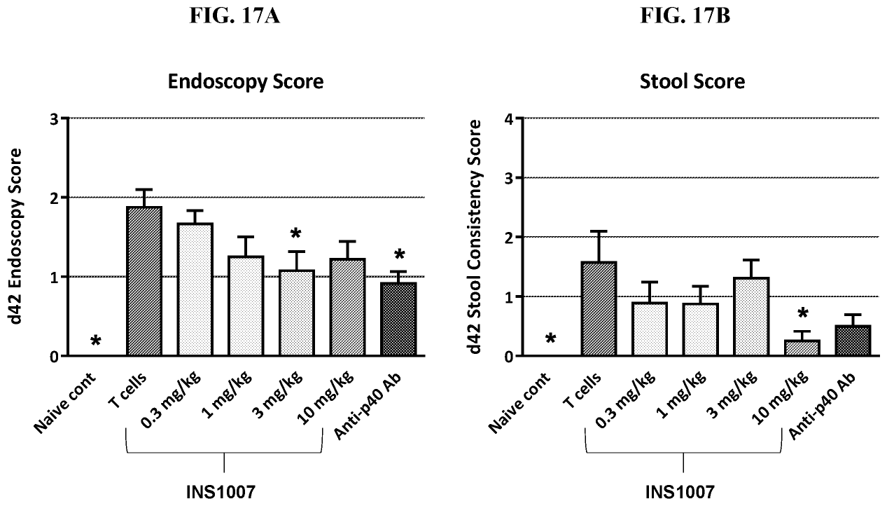

Mean Endoscopy Score: Animals underwent video endoscopy on Days 14, 28, and 42 to assess the severity of colitis in each treatment group. Mean endoscopy scores for Days 28 and 42 are presented in FIGS. 16A and 17A, respectively.

On all days of evaluation, colitis was observed in all groups that received adoptive transfer (Groups 2-7) compared to naïve animals (Group 1).

On Day 14, a reduction of 22.2% as compared to vehicle control (Group 2) mean endoscopy score was observed for the 3 mg/kg INS1007 group (Group 5).

At Day 28, the mean endoscopy score was significantly elevated in vehicle control (Group 2) compared to naïve animals (Group 1) (FIG. 16A), which indicates successful colitis induction. Also at Day 28, reductions in mean endoscopy score were observed in the 1 mg/kg INS1007 (38.4%, Group 4), 3 mg/kg INS1007 (39.5%, Group 5), 10 mg/kg INS1007 (15.3%, Group 6) and anti-p40 g (27.4%, Group 7) treatment groups as compared to vehicle control (Group 2).

At Day 42, the mean endoscopy score was significantly elevated in vehicle control (Group 2) compared to naïve animals (Group 1) (FIG. 17A), which sustained T-cell induced colitis. Also at Day 42, reductions in mean endoscopy score were observed in the 1 mg/kg INS1007 (33.5%, Group 4), 3 mg/kg INS1007 (42.7%, Group 5), 10 mg/kg INS1007 (35.0%, Group 6) and anti-p40 g (51.24%, Group 7) treatment groups as compared to vehicle control (Group 2).

Stool consistency: Animals underwent video endoscopy on Days 14, 28, and 42 to assess the stool consistency in each treatment group. Mean stool consistency scores for Days 28 and 42 are presented in FIGS. 16B and 17B, respectively.

At Day 14, the mean stool consistency was significantly elevated in vehicle control (Group 2) compared to naïve animals (Group 1) (FIGS. 16A and 17A) and all other treatment groups (FIG. 16B). The mean stool consistency score for Groups 1 and 3-7 was 0 at this timepoint.

At Day 28, reductions in the mean stool consistency score were observed for 1 mg/kg INS1007 (Group 4), 3 mg/kg INS1007 (Group 5), 10 mg/kg INS1007 (Group 6) and anti-p40 g (Group 7) treatment groups as compared to vehicle control (Group 2).

At Day 42, reductions in mean stool consistency scores were observed for 1 mg/kg INS1007 (Group 4), 3 mg/kg INS1007 (Group 5), 10 mg/kg INS1007 (Group 6) and anti-p40 g (Group 7) treatment groups as compared to vehicle control (Group 2). The largest reductions were observed in the 10 mg/kg INS1007 (Group 6) and anti-p40 g (Group 7) treatment groups.

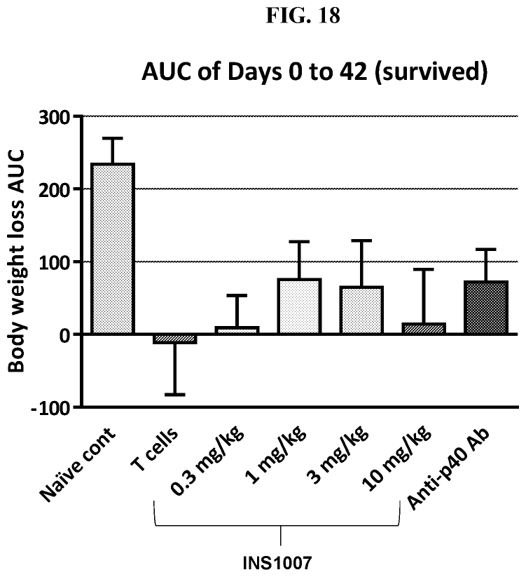

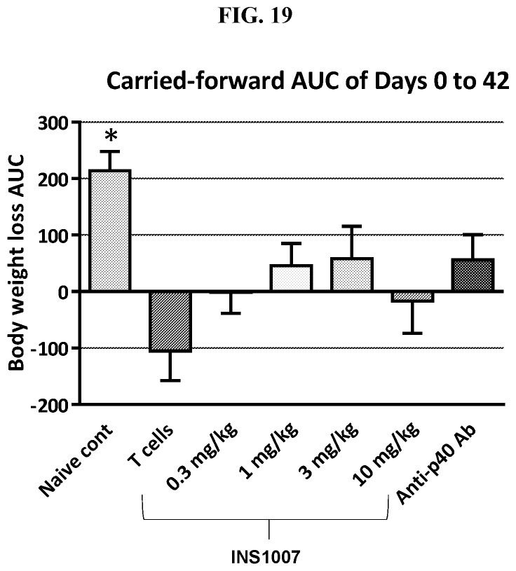

Weight Loss: Weight loss calculated as a percentage of the starting weight on Day 0 was recorded in all treatment groups. FIG. 18 shows the body weight loss area under the curve (AUC, calculated using the trapezoidal transformation rule) for mice surviving to Day 42. To account for any potential survivor bias, the body weight loss AUC was calculated for on all study animals (i.e., those surviving to Day 42 and those that did not) by carrying forward the body weight at death for animals that did not survive to Day 42 (Carried-Forward AUC). The Carried-Forward AUC results are shown in FIG. 19.

Naïve animals (Group 1) gained weight over the duration of the study. In contrast, animals with adoptive transfer T-cell induced colitis showed variable body weight gain and loss if the body weights for animals that were euthanized and/or found dead prior to Day 42 were not carried forward (FIG. 18). Carrying forward the body weight for deceased animals (FIG. 19), animals with adoptive transfer T-cell induced colitis showed body weight loss beginning on Day 21. The body weight loss was greatest in vehicle control (Group 2). Compared to vehicle control (Group 2), weight loss was reduced in 3 mg/kg INS-1007 (Group 5) and anti-p40 (Group 7) treatment groups. In general, all treated groups (Groups 3-7) demonstrated less pronounced body weight loss as compared to vehicle control (Group 2).

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

### REFERENCES

¹ Abraham C, Cho J H. Inflammatory bowel disease. N Engl J Med. 2009; 361:2066-78
² About Crohn's disease (2009). Crohn's & Colitis Foundation of America. Available at: http://www.ccfa.org/info/about/crohns; Crohn's disease. National Institute of Diabetes and Digestive and Kidney Diseases. http://digestive.niddk.nih.gov/ddiseases/pubs/crohns/Crohns.pdf
³ A. Mizoguchi, et al., Animal models of IBD: linkage to human disease. Curr Opin Pharmacol. 2010; 10:578-87.
⁴ D. Ostanin, et al., T cell transfer model of chronic colitis: concepts, considerations, and tricks of the trade. Am. J. Physiol. Gastrointest. Liver Physiol. 2009; 296(2):G135-46.
⁵ Crohn's disease. National Institute of Diabetes and Digestive and Kidney Diseases. http://digestive.niddk.nih.gov/ddiseases/pubs/crohns/Crohns.pdf, Management of Crohn's disease in adults. Bethesda, Md.: American College of Gastroenterology. http://www.acg.gi.org/physicians/guidelines/CrohnsDiseaseinAdults-2009.pdf
⁶ Management of Crohn's disease in adults. Bethesda, Md.: American College of Gastroenterology. http://www.acg.gi.org/physicians/guidelines/CrohnsDiseaseinAdults-2009.pdf, Ford A C, Sandborn W J, Khan K J, Hanauer S B, Talley N J, Moayyedi P. Efficacy of biological therapies in inflammatory bowel disease: Systematic review and meta-analysis. American Journal of Gastroenterology. 2011; 106:644-59.

1. A method for treating inflammatory bowel disease (IBD) in a patient in need of treatment, comprising, administering to the patient a pharmaceutical composition comprising an effective amount of a compound of formula (I), or a pharmaceutically acceptable salt thereof, 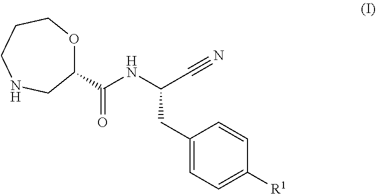
wherein,
R¹ is 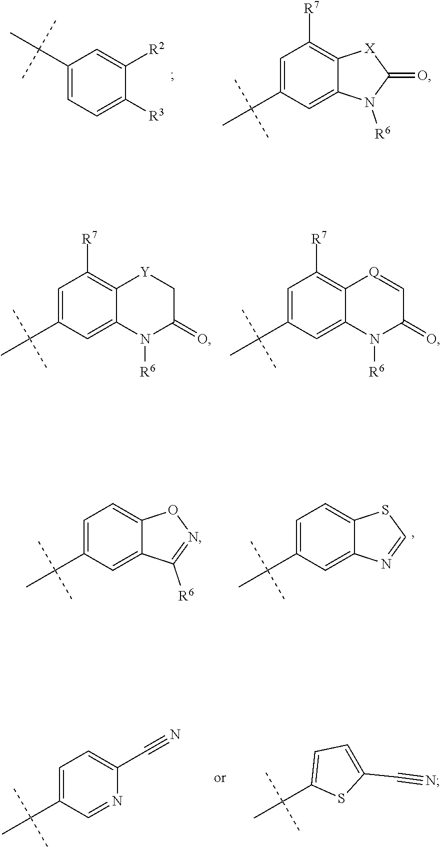
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

2. The method of claim 1, wherein, R¹ is

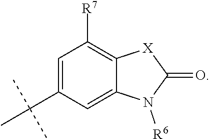

3. The method of claim 1 or claim 2, wherein, X is O; R⁶ is C₁₋₃alkyl; and R⁷ is hydrogen.

4. The method of claim 1, wherein the compound of formula (I) is selected from the group consisting of
(2S)—N-[(1S)-1-Cyano-2-(4′-cyanobiphenyl-4-yl)ethyl]-1,4-oxazepane-2-carboxamide;
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide;
4′-[(2S)-2-Cyano-2-{[(2S)-1,4-oxazepan-2-ylcarbonyl]amino}ethyl]biphenyl-3-yl methanesulfonate;
(2S)—N-{(1S)-1-Cyano-2-[4-(3-methyl-1,2-benzoxazol-5-yl)phenyl]ethyl}-1,4-oxazepane-2-carboxamide;
(2S)—N-{(1S)-1-Cyano-2-[4′-(trifluoromethyl)biphenyl-4-yl]ethyl}-1,4-oxazepane-2-carboxamide;
(2S)—N-[(1S)-1-Cyano-2-(3′,4′-difluorobiphenyl-4-yl)ethyl]-1,4-oxazepane-2-carboxamide;
(2S)—N-{(1S)-1-Cyano-2-[4-(6-cyanopyridin-3-yl)phenyl]ethyl}-1,4-oxazepane-2-carboxamide;
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide;
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide;
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide;
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide;
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide;
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide;
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide;
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide;
(2S)—N-{(1S)-1-Cyano-2-[4-(5-cyanothiophen-2-yl)phenyl]ethyl}-1,4-oxazepane-2-carboxamide;
(2S)—N-[(1S)-2-(4′-Carbamoyl-3′-fluorobiphenyl-4-yl)-1-cyanoethyl]-1,4-oxazepane-2-carboxamide;
(2S)—N-{(1S)-1-Cyano-2-[4-(1-methyl-2-oxo-1,2-dihydroquinolin-7-yl)phenyl]ethyl}-1,4-oxazepane-2-carboxamide;
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide;
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide;
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide;
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide;
(2S)—N-{(1S)-1-Cyano-2-[4′-(methylsulfonyl)biphenyl-4-yl]ethyl}-1,4-oxazepane-2-carboxamide;
(2S)—N-{(1S)-2-[4′-(Azetidin-1-ylsulfonyl)biphenyl-4-yl]-1-cyanoethyl}-1,4-oxazepane-2-carboxamide;
(2S)—N-[(1S)-1-Cyano-2-(4′-fluorobiphenyl-4-yl)ethyl]-1,4-oxazepane-2-carboxamide;
(2S)—N-{(1S)-2-[4-(1,3-Benzothiazol-5-yl)phenyl]-1-cyanoethyl}-1,4-oxazepane-2-carboxamide; or
(2S)—N-[(1S)-1-Cyano-2-(4′-cyanobiphenyl-4-yl)ethyl]-1,4-oxazepane-2-carboxamide;
and pharmaceutically acceptable salts thereof.

5. The method of claim 1, wherein the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide: 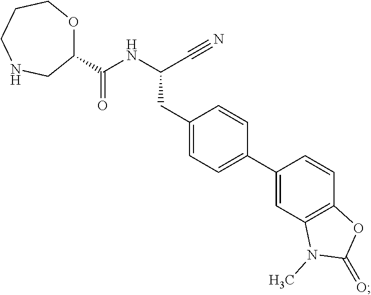 [SMILES: Cn1c(=O)oc2ccc(-c3ccc(C[C@@H](C#N)NC(=O)[C@@H]4CNCCCO4)cc3)cc21]
or a pharmaceutically acceptable salt thereof.

6. The method of claim 1, wherein the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide.

7. The method of any one of claims 1-6, wherein the composition comprises a pharmaceutically acceptable adjuvant, diluent or carrier.

8. The method of any one of claims 1-7, wherein administering comprises oral administration.

9. The method of any one of claims 1-8, wherein administering to the patient is carried out one time daily.

10. The method of any one of claims 1-8, wherein administering to the patient is carried out two times daily.

11. The method of any one of claims 1-8, wherein administering to the patient is carried out once, every other day.

12. The method of any one of claims 1-8, wherein administering to the patient is carried out once every third day.

13. The method of any one of claims 1-12, wherein the patient's fecal calprotectin (FCP) level is greater than about 250 μg/g.

14. The method of any one of claims 1-13, wherein the patient's serum c-reactive protein (CRP) level is greater than about 5 mg/dL.

15. The method of any one of claims 1-14, wherein the IBD is Crohn's disease.

16. The method of claim 15, wherein the Crohn's disease is moderately to severely active Crohn's disease.

17. The method of claim of claim 16, wherein the patient's Crohn's Disease Activity Index (CDAI) is greater than or equal to 220 and less than or equal to 450.

18. The method of claim 16, wherein the patient's Harvey Bradshaw Index score is greater than or equal to 7.

19. The method of any one of claims 1-14, wherein the IBD is ulcerative colitis.

20. The method of claim 19, wherein the ulcerative colitis is moderately to severely active ulcerative colitis.

21. The method of claim 20, wherein the patient's total Mayo score is greater than about 6.

22. The method of claim 21, wherein the patient's Mayo endoscopic subscore is greater than about 2.

23. The method of any one claims 1-22, wherein the treating comprises decreasing the FCP levels as compared to the patient's FCP level prior to treatment.

24. The method of any one of claims 1-22, wherein the treating comprises decreasing the serum patient's CRP levels as compared to the patient's CRP levels prior to treatment.

25. The method of any one of claims 1-18 and 23-24, wherein the treating comprises decreasing the patient's CDAI score as compared to the CDAI score prior to treatment.

26. The method of claim 25, wherein the treating comprises decreasing the patient's CDAI score by at least about 100 points as compared to the CDAI score prior to treatment.

27. The method of claim 25, wherein the treating comprises decreasing the patient's CDAI score to less than about 150 points.

28. The method of any one of claims 1-18 and 23-24, wherein the treating comprises decreasing the patient's Simplified Endoscopy Score for Crohn's Disease (SES-CD) score as compared to the SES-CD score prior to treatment.

29. The method of claim 28, wherein the treating comprises decreasing the patient's SES-CD score to less than about 4.

30. The method of any one of claims 1-18 and 23-24, wherein the treating comprises achieving endoscopic improvement in said patient as compared to prior to treatment.

31. The method of any one of claims 1-18 and 23-24, wherein the treating comprises decreasing the patient's Harvey Bradshaw Index score as compared to the Harvey Bradshaw Index score prior to treatment.

32. The method of any one of claims 1-18 and 19-24, wherein the treating comprises decreasing the patient's total Mayo score as compared to the total Mayo score prior to treatment.

33. The method of any one of claims 1-18 and 19-24, wherein the treating comprises decreasing at least one of the patient's Mayo subscores selected from stool frequency subscore, rectal bleeding subscore, physician's global assessment and endoscopy subscore as compared to the patient's Mayo subscore prior to treatment.

34. The method of any one of claims 32 and 33, wherein the treating comprising decreasing the patient's total Mayo score by at least about 3 points as compared to the total Mayo score prior to treatment.

35. The method of any one of claims 32-34, wherein the treating comprising decreasing the patient's total Mayo score by at least about 30% as compared to the total Mayo score prior to treatment.

36. The method of any one of claims 1-18 and 19-24, wherein the treating comprises decreasing the patient's total Ulcerative Colitis Activity Index (UCDAI) score as compared to the total UCDAI score prior to treatment.

37. The method of claim 36, wherein the treating comprising decreasing the patient's total UCDAI score to less than about 1.

38. The method of any one of claims 36 and 37, wherein the treating comprises decreasing at least one of the patient's UCDAI subscores selected from stool frequency subscore, rectal bleeding subscore, physician's rating of disease activity and mucosal appearance subscore as compared to the patient's Mayo subscore prior to treatment.

39. The method of any one of claims 1-18 and 19-24, wherein the treating comprises mucosal healing of the patient's Ulcerative Colitis as compared to prior to treatment.

40. The method of claim 39, wherein the treating comprises decreasing the patient's Mayo endoscopy subscore to one or less.

41. The method of claim 39, wherein the treating comprises decreasing the patient's histological grade to zero according to the Geboes histological assessment.

42. The method of any one of claims 1-41, wherein the treating comprises achieving remission of the patient's IBD.

43. The method of claim 42, wherein the treating comprises achieving sustained remission of the patient's IBD.

44. The method of any one of claims 42 and 43, wherein the treating comprises decreasing the patient's CDAI score to less than about 150 points.

45. The method of any one of claims 42 and 43, wherein the treating comprises decreasing the patient's Harvey Bradshaw Index score to less than 4 points.

46. The method of any one of claims 42 and 43, wherein the treating comprises decreasing the patient's total Mayo score to less than or equal to 2 points with no subscore greater than 1 point.

47. The method of any one claims 42 and 43, wherein the treating comprises decreasing the FCP levels to less than about 250 μg/g.

48. The method of any one of claims 42 and 43, wherein the treating comprises decreasing the serum patient's CRP levels to less about 5 mg/dL.

49. The method of any one of claims 1-48, further comprising administering one or more additional active agents to the patient in need of treatment.

50. The method of claim 49, wherein the one or more additional active agents comprises an anti-inflammatory drug.

51. The method of claim 50, wherein the anti-inflammatory drug is selected from the group consisting of sufasazine, mesalamine, balsalazide, olsalazine and one or more corticosteroids.

52. The method of claim 49, wherein the one or more additional active agents comprises an immune system modulator.

53. The method of claim 52, wherein the immune system modulator is selected from the group consisting of azathioprine, 6-mercaptopurine, methotrexate, an anti-tumor necrosis factor α (TNF-α) monoclonal antibody, and an anti-p40 monoclonal antibody.

54. The method of claim 53, wherein the anti-p40 monoclonal antibody comprises ustekinumab.

55. The method of 53, wherein the anti-TNF-α monoclonal antibody is selected from the group consisting of infliximab, adalimumab, and certolizumab pegol.
